(19) United States
(12) Patent Application Publication
(10) Pub. No.: US 2023/0355792 A1
(43) Pub. Date: Nov. 9, 2023

(54) METHODS OF IMPROVING THE THERAPEUTIC INDEX

(71) Applicant: Heidelberg Pharma Research GmbH, Ladenburg (DE)

(72) Inventors: Michael KULKE, Ladenberg (DE); Torsten HECHLER, Ladenburg (DE); Kristin DECKER, Ladenburg (DE); Andreas PAHL, Ladenburg (DE)

(73) Assignee: Heidelberg Pharma Research GmbH, Ladenburg (DE)

(21) Appl. No.: 18/296,452

(22) Filed: Apr. 6, 2023

(30) Foreign Application Priority Data
Apr. 7, 2022 (EP) 22167261.1

(51) Int. Cl. A61K 47/68

(52) U.S. Cl. CPC A61K 47/6831; A61K 47/6851

(57) ABSTRACT

The present invention pertains to treatment regimens to increase the therapeutic index of antibody-drug conjugates in particular antibody-drug conjuagtes comprising amatoxins. The present invention furthermore pertains to amatoxin-based antibody-drug conjuagtes for use in said treatment regimens.

### INCORPORATION-BY-REFERENCE OF MATERIAL ELECTRONICALLY SUBMITTED

Incorporated by reference in its entirety herein is a computer-readable nucleotide/amino acid sequence listing submitted concurrently herewith and identified as follows: 4,251 bytes Extensible Markup Language (XML) file named “767171_ST26_SequenceListing.xml” created Jul. 5, 2023.

### FIELD OF THE INVENTION

The present application relates to the field of cancer immunotherapy. In particular, the present application relates to dosing regimens for the treatment of cancer in a patient to increase the therapeutic index of amatoxin-based antibody drug-conjugates. The present application further relates to amatoxin-based antibody drug-conjugates for use in such treatment regimes.

### BACKGROUND

Currently approved antibody-drug conjugates (ADCs) such as Enhertu (HER2), Trodelvy (Trop-2) and Blenrep (BCMA)) are based on only a few cytotoxic mechanisms with mainly microtubule- or DNA-targeting toxins as payloads. Amatoxins are a well-known group of toxins consisting of nine structurally related toxins naturally occurring in basidiomycetes mushrooms of the genus Amanita. The most prominent members are alpha and beta-amanitin identified more than forty years ago in the green death cap mushroom Amanita Phalloides. Amatoxins are well known to the public and have been intensively studied in healthcare and medical science due to their toxic effects after mushroom ingestion. They are responsible for 95% of all fatal mushroom intoxications world-wide. Amatoxins are highly heat resistant, resistant to enzymatic and acidic degradation and show an excellent solubility in water. Especially alpha-amanitin has been shown to be mainly responsible for severe liver and kidney injury observed after A. phalloides poisoning. In the liver it is taken up by a specific transporter, the organic anion transporting polypeptide (OATP1B3), located on hepatocytes leading to their cytolysis.

Due to its hydrophilic nature amanitin does not passively penetrate cells and no or only poor uptake has been shown for most other human cell types except for hepatocytes. However, it has been reported that beside the liver other organs, mainly the kidney, are also affected. Since Amatoxins are not further metabolized, they are mainly excreted through the kidney and the urine within the first days after ingestion, leading to severe nephrotoxicity.

Amatoxins are bicyclic octapeptides comprising the three unusual amino acids dihydroxyisoleucine, hydroxy-trypto- phan and hydroxy-proline. They bind with highest affinity to eukaryotic RNA Polymerase II resulting in a dramatic over 1000-fold decrease in transcription and protein synthesis. The OH-groups of the hydroxylated side chains are responsible for the high hydrophilicity of the molecule and to a certain degree for its engagement to the RNA Polymerase II. Due to this highly specific inhibition amanitin has been widely used as a tool to investigate gene expression and transcriptional regulation.

Importantly the amanitin-induced effects are not restricted to dividing cells and work also in resting (non-dividing) cells, unlike toxins which target DNA or Tubulin which are currently used in most approved ADCs.

Through this novel mode of action, amanitin-based ADCs have thus several advantages compared to marketed ADCs: Firstly, amanitin-based ADCs also affect non-proliferating and quiescent cells. Secondly, amanitin is not a known substrate to efflux transporters. Thus, amanitin-based ADCs are also efficacious in tumors that are resistant to other lines of therapy. Thirdly, there are no known resistance mechanisms to amanitin as ADC payload which is likely due to a strict structure-activity requirement of RNA polymerase II in eukaryotic cells that provides virually no room for escape mutations in RNA polymerase II.

Amatoxins are well suited based on their physico-chemical properties to be used as a payload for an ADC: First, amatoxins allow the attachment ofa linker without diminishing its cytotoxic potential, second, amatoxins possess a high cytotoxic potential to achieve cell killing at low concentrations and third, when coupled to an antibody, amatoxins unfold their cytotoxic potential only inside a cell.

In addition to its promising efficacy profile, amanitin shows some favorable physicochemical properties. In contrast to all other toxins typically used as ADC payloads (warheads), the molecule is very hydrophilic. This facilitates conjugation in aqueous buffers and prevents aggregation of the ADC, which makes it easy to handle and lowers its potential for immunogenicity. It has also been shown that amanitin is a very poor substrate for the initiation of multi drug resistance (MDR) processes, and that amanitin conjugates are highly potent in MDR-expressing tumour cells.

Amanitin is highly stable in plasma and due to its low molecular weight and hydrophilicity it is cleared very rapidly via the kidney, which makes it unlikely to be accumulated in other tissues. Another safety feature is its inability to enter normal cells via passive uptake. The free toxin shows a 20,000-fold reduced cytotoxicity compared to an amanitin-conjugated antibody., Amanitin or amatoxins when conjugated to an antibody are no longer a substrate for OATP1B3-mediated cellular uptake which reduces in particular liver toxicity, however, some liver toxicity can be observed in animal models as evidenced by a transient increase in liver enzymes such as ALT, AST or LDH upon administration of amatoxin-based ADCs which reduces the therapeutic window of such amatoxin-based ADCs. Thus, there is sill a continued medical need to further improve the tolerability and thereby the therapeutic index or therapeutic window of amatoxin-based ADCs.

### SUMMARY OF THE INVENTION

As shown in the present application, the inventors unexpectedly found that by administering a first sub-therapeutic dose of a first amatoxin-based ADC prior to administering a therapeutic dose of a second amatoxin-based ADC the liver toxicity of the second amatoxin-based ADC can be further reduced resulting in an increased therapeutic index of the second amatoxin-based ADC. The Inventors further surpsingly found that this effect could also be achieved if the first and second amatoxin-based ADC did bind to different target proteins, as demonstrated through the use of a non-binding antibody for said first ADC of the inventive method.

It hence is one object of the present invention to provide a method of improving the therapeutic index of an antibody-drug-conjugate, wherein the inventive method comprises the step of administerig to a patient a first dose of a first antibody-drug-conjugate, wherein said first dose of said first antibody-drug conjugate is administered between about 30 days to about 15 days prior to the administration of a second dose of an antibody-drug conjugate and wherein said antibody-drug conjugate has the structure of

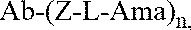

wherein an antibody or antigen-binding fragment thereof (Ab) is conjugated (covalently linked) to linker (L), through a chemical moiety (Z), to an amatoxin moiety (Ama) and n is from about 1, 2, 3, 4 to about 5, 6, 7, 8.

The present invention further provides for a first and/or amatoxin-based ADCs as disclosed herein for use in the inventive method to improve the therapeutic index of said second amatoxin-based ADC, whereby the first and second amatoxin-based ADC may be identical, or may be different form each other.

### DESCRIPTION OF THE FIGURES

FIG. 1. Structural formulae of various amatoxins (SEQ ID NOs:1 and 2). The numbers in bold type (1 to 8) designate the standard numbering of the eight amino acids forming the amatoxin. The standard designations of the atoms in amino acids 1, 3, and 4 are also shown (Greek letters α to γ, Greek letters α to δ, and numbers from 1′ to 7′, respectively).

FIG. 2 (A) Experimental study design of study in CB17-SCID mice with and without amatoxin-based ADAC pre-treatment. (B) Two independent experiments with 3 (upper panel) or 5 (lower panel) mice/group were performed using different doses of the anti-DIG amatoxin-ADC. Number of mice with macroscopic changes in the liver as well as survival is depicted; due to autolytic state, not all mice could be examined for liver abnormalities. p-value was calculated using a Gehan-Breslow-Wiloxon-test.

FIG. 3 Repeated dosing reduces amatoxin-ADC-induced organ damage of in Cynomolgus monkeys. (A) Experimental set-up comparing the toxicity of a single dose with the maximal tolerated dose (MTD) of amatoxin-ADC (left) with the toxicity occurring after the same dose was given as final step of a dose escalation scheme. (B) aspartate aminotransferase (AST), Alanine aminotransferase (ALT) and Lactatdehydrogenase (LDH) levels in the serum of Cynomolgus monkeys following i.v. treatment with amatoxin-ADC either given as single dose (circles) or as final step of a dose escalation scheme (triangles). Dotted lines: baseline levels in untreated animals. Mean of three biological replicates with SEM is shown. p-value: Welch’s t-test; *: p ≤ 0.05. (C) Experimental set-up comparing toxicity of the MTD of amatoxin-ADC given after different rounds of treatment. (D) AST, ALT and LDH levels in the serum of cynomolgus monkeys following i.v. treatment with the MTD of amatoxin-ADC given as first (triangles), second (open circles), third (open triangle) or fourth (polygon) treatment round. Depicted is the mean of three biological replicates with SEM.

FIG. 4 Repeated dosing with an amatoxin-based anti-PSMA ADC improves the anti-tumor efficacy in a s.c. LNCaP CDX model (A) Experimental set-up of an in vivo efficacy study in a s.c. human prostate adenocarcinoma LNCaP CDX model comparing single dose with multiple dose (once weekly for 4 weeks, q7dx4) treatment of an amatoxin-based anti-PSMA ADC; (B) tumor growth of s.c. LNCaP tumors measured by caliper for 84 days (n = 18 animals/group), triangles:single dose treatment, squares: multiple dose treatment, filled circles: PBS control. Means with SD are shown. p-value: Welch t-test with multiple testing correction by Holm-Sídák Method; statistically significant from day 21 to 56. ** p < 0.01; (C) survival probability of mice carrying LNCaP s.c. tumors which were either treated with a single dose (triangles) or multiple doses (squares) of the amatoxin-based anti-PSMA ADC; circles: PBS control. p-value: Gehan-Breslow-Wiloxon-test; ****: p < 0.001

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X + Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention. The word “substantially” does not exclude “completely” e.g., a composition which is “substantially free” from Y may be completely free from Y. Where necessary, the word “substantially” may be omitted from the definition of the invention.

The term “about” in relation to a numerical value x as used herein means x ± 10%.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will supersede any other definition. In addition, the materials, methods, and examples are illustrative only and not intended to be limiting.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure and avoid lengthy repetitions. Chemical terminology used throughout the present application have according to the “Compendium of Chemical Terminology” published by the International Union of Pure and Applied Chemistry, ISBN: 0-9678550-9-8.

According to a first aspect, the present invention relates to a method of increasing the therapeutic index of an antibody-drug-conjugate, wherein the method comprises the step of administerig to a patient afflicted with cancer a first dose of a first antibody-drug-conjugate, wherein said first dose of said first antibody-drug conjugate is administered between about 30 days to about 15 days prior to the administration of a second dose of an antibody-drug conjugate.

Accordingly, in a first aspect, the present invention relates to a method of increasing the therapeutic index of an antibody-drug-conjugate, wherein the method comprises the step of administerig to a patient afflicted with cancer a first dose of a first antibody-drug-conjugate, wherein said first dose of said first antibody-drug conjugate is administered between about 30 days to about 15 days prior to the administration of at least a second dose of an antibody-drug conjugate.

According to one embodiment, the first and second dose of the antibody-drug conjugate (ADC) in the method according to the invention comprises the same antibody-drug conjugate. The term “therapeutic index” as used herein refers to the ratio of toxic dose at which 50% of the individuals show toxic effects of a drug to the minimal concentration or amount of a drug at which 50% of the individuals show therapeutic effect. The TI may e.g. also be expressed as TI=TD₅₀:ED₅₀ and is a quantitative measurement of the relative safety of a drug, whereby TD₅₀refers to the median toxic dose of a drug or toxin, which is the dose at which toxicity occurs in 50% of cases, and ED₅₀ refers to the median effective dose which is the dose that produces a quantal effect (all or nothing) in 50% of the individuals receiving a given drug, such as the amatoxin-based ADCs for use according to the invention. It is a comparison of the amount of a therapeutic agent that causes the therapeutic effect to the amount that causes toxicity. Accordingly, a greater TI corresponds to an increased relative safety of a given drug. In some aspects, the therapeutic index can include a comparison of the amount of a therapeutic agent, such as the first and/or second ADC as disclosed herein, that causes the therapeutic effect (e.g. killing cancer cells) to the amount of the therapeutic agent that causes toxicity (e.g., liver toxicity). Accordingly, increasing the therapeutic index of the first and/or second ADC according to the invention is advantageous as it increases the safety of the respective ADC, preferably the inventive method is used to increase the therapeutic index of said second ADC.

In one aspect, the invention pertains to a first and/or second ADC for use in the inventive method of increasing the therapeutic index of said first and/or second ADC, preferably of the first and/or second ADC as disclosed herein for use according to the invention to increase the the therapeutic index of said second ADC as disclosed herein.

In some embodiments, the inventions pertains to a first and/or second ADC for use in a method of increasing the therapeutic index of an antibody-drug-conjugate, wherein the method comprises administerig to a patient afflicted with cancer a first dose of a first antibody-drug-conjugate, wherein said first dose of said first antibody-drug conjugate is administered between about 30 days to about 15 days prior to the administration of at least second dose of an antibody-drug conjugate.

The term “same ADC” as used in accordance with the inventive method refers to the fact that the first and second ADC for use in the inventive method are identical molecules. For example, the first and second ADC of the inventive method may be identical in terms of their composition (e.g. antibody, linker, payload) and may e.g. be formulated into a pharmaceutical composition having the same composition. The term “pharmaceutical composition” as used herein means a product comprising pharmaceutical excipients such as buffering agents, preservatives and tonicity modifiers together with the active compound such as e.g. amatoxin-based ADCs as disclosed herein. The term “administering” or any grammatical equivalent thereof refers to implanting, absorbing, ingesting, injecting, inhaling, or otherwise introducing a compound, such as e.g. the amatoxin-based ADCs of the invention as disclosed herein, or a corresponding pharmaceutical composition comprising said ADCs, preferably, admininistering refers to injecting, more preferably to intravenous (i.v.) or subcutaneous (s.c.) injection of a first and/or second amatoxin-based ADC as disclosed herein.

In some instances, it may, e.g. be advantageous if the first and second amatoxin-based ADC for use in the inventive method as disclosed herein are identical to reduce the risk of adverse effects as only one ADC is administered.

The term “cancer” as used with the inventive method refers to breast cancer, including triple-negative breast cancer, biliary tract cancer; bladder cancer; brain cancer including glioblastomas and medulloblastomas; cervical cancer; choriocarcinoma; colon cancer; endometrial cancer; esophageal cancer; gastric cancer; gastrointestinal stromal tumor (GIST), appendix cancer, cholangiocarcinoma, carcinoid tumor, gastrointestinal colon cancer, extrahepatic bile duct cancer, gallbladder cancer, gastric (stomach) cancer, gastrointestinal carcinoid tumor, colorectal cancer, or metastatic colorectal cancer, hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia lymphoma; non-Hodgkin’s lymphoma (NHL), follicular lymphoma, diffuse large B cell non-Hodgkin’s lymphoma (DBNHL), subtypes of non-Hodgkin’s lymphoma including mantle cell lymphoma (MCL), chronic lymphocytic leukaemia (CLL), Richter syndrome, primary cutaneous marginal zone lymphoma (PCMZL), hairy cell leukemia, acute myeloid leukemia (AML), rheumatoid arthritis, granulomatosis with polyangiitis and microscopic polyangiitis and pemphigus vulgaris, intraepithelial neoplasms including Bowen’s disease and Paget’s disease; liver cancer; lung cancer, including non-small cell lung cancer, lymphomas including Hodgkin’s disease and lymphocytic lymphomas; neuroblastomas; glioblastoma, oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi’s sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor.

In one embodiment of the present invention, the first and second ADC may be different. For example, the first ADC according to the invention may bind to a target on a tumor cell, the second ADC according to the invention may comprise an antibody moiety that does not specifically bind to a target in the patient to whom the ADC is administered. The term “specific binding” or any grammatical equivalent thereof ”, antibody/antigen, or other binding pair, indicates a binding reaction which is determinative of the presence of the protein in a heterogeneous population of proteins and other biologics. Thus, a given antibody, or binding composition derived from the antigen-binding site of an antibody, of the contemplated method binds to its antigen with an affinity that is at least two fold greater, preferably at least ten times greater, more preferably at least 20-times greater, and most preferably at least 100-times greater than the affinity with unrelated antigens. For example, ADCs that do not specifically bind to a target in the patient to whom the ADC is adminsteredd may be directed to non-human target antigens, such as e.g. digoxigenin (anti-DIG, e.g. as disclosed in WO2022/045433A1), hen egg lysozyme (anti-HEL), green fluorescent protein (GFP) and related proteins such as those disclosed in US7,906,636 B2, or any human or humanized antibody which does not specifically bind to a cell surface antigen in a patient. In preferred embodiments, the first and/or second ADC comprises a humanized or human antibody moiety, which may also be referred to as antibody. The use of humanized or human antibody moieties (antibodies) in said first and/or second ADC is advantageous as it reduced the immunogencicity of said ADCs and the likelihood of antibody-drug-antibodies.

According to one embodiment, the first and second ADC according to the invention comprise an RNA polymerase II inhibitor. According to preferred embodiments, the RNA polymerase II inhibitor of the invention is selected from the group of amatoxins. In the context of the present invention the term “amatoxin” includes all cyclic peptides composed of 8 amino acids as isolated from the genus Amanita and described in Wieland, T. and Faulstich H. (Wieland T, Faulstich H., CRC Crit Rev Biochem. 5 (1978) 185-260), further all chemical derivatives thereof; further all semisynthetic analogs thereof; further all synthetic analogs thereof built from building blocks according to the master structure of the natural compounds (cyclic, 8 amino acids), further all synthetic or semisynthetic analogs containing non-hydroxylated amino acids instead of the hydroxylated amino acids, further all synthetic or semisynthetic analogs, in which the sulfoxide moiety is replaced by a sulfone, thioether, or by atoms different from sulfur, e.g., a carbon atom as in a carbanalog of amanitin.

As used herein, a “derivative” of a compound refers to a species having a chemical structure that is similar to the compound, yet containing at least one chemical group not present in the compound and/or deficient of at least one chemical group that is present in the compound. The compound to which the derivative is compared is known as the “parent” compound. Typically, a “derivative” may be produced from the parent compound in one or more chemical reaction steps.

As used herein, an “analogue” of a compound is structurally related but not identical to the compound and exhibits at least one activity of the compound. The compound to which the analogue is compared is known as the “parent” compound. The afore-mentioned activities include, without limitation: binding activity to another compound; inhibitory activity, e.g. enzyme inhibitory activity; toxic effects; activating activity, e.g. enzyme-activating activity. It is not required that the analogue exhibits such an activity to the same extent as the parent compound. A compound is regarded as an analogue within the context of the present application, if it exhibits the relevant activity to a degree of at least 1% (more preferably at least 5%, more preferably at least 10%, more preferably at least 20%, more preferably at least 30%, more preferably at least 40%, and more preferably at least 50%) of the activity of the parent compound. Thus, an “analogue of an amatoxin”, as it is used herein, refers to a compound that is structurally related to any one of α-amanitin, β-amanitin, γ- amanitin, ε-amanitin, amanin, amaninamide, amanullin, and amanullinic acid and that exhibits at least 1% (more preferably at least 5%, more preferably at least 10%, more preferably at least 20%, more preferably at least 30%, more preferably at least 40%, and more preferably at least 50%) of the inhibitory activity against mammalian RNA polymerase II as compared to at least one of α-amanitin, β-amanitin, γ-amanitin, ε-amanitin, amanin, amaninamide, amanullin, and amanullinic acid. An “analogue of an amatoxin” suitable for use in the present invention may even exhibit a greater inhibitory activity against mammalian RNA polymerase II than any one of α-amanitin, β-amanitin, γ-amanitin, ε-amanitin, amanin, amaninamide, amanullin, or amanullinic acid. The inhibitory activity can be measured by determining the concentration at which 50% inhibition occurs (IC₅₀ value). The inhibitory activity against mammalian RNA polymerase II can be determined indirectly by measuring the inhibitory activity on cell proliferation, as e.g. disclosed in Voss et al. BMC Molecular Biology 2014, 15:7. A “semisynthetic analogue” refers to an analogue that has been obtained by chemical synthesis using compounds from natural sources (e.g. plant materials, bacterial cultures, fungal cultures or cell cultures) as starting material. Typically, a “semisynthetic analogue” of the present invention has been synthesized starting from a compound isolated from a mushroom of the Amanitaceae family. In contrast, a “synthetic analogue” refers to an analogue synthesized by so-called total synthesis from small (typically petrochemical) building blocks. Usually, this total synthesis is carried out without the aid of biological processes.

According to some embodiments,, the amatoxin of said first and/or second ADC for use according to the invention is be selected from the group consisting of α-amanitin, β-amanitin, γ-amanitin, ε-amanitin, amanin, amaninamide, amanullin, amanullinic acid, and analogues, derivatives and salts thereof.

Functionally, amatoxins are defined as peptides or depsipeptides that inhibit mammalian RNA polymerase II. Preferred amatoxins are those with a functional group (e.g. a carboxylic group, an amino group, a hydroxy group, a thiol or a thiol-capturing group) that can be reacted with linker molecules or target-binding moieties as defined below.

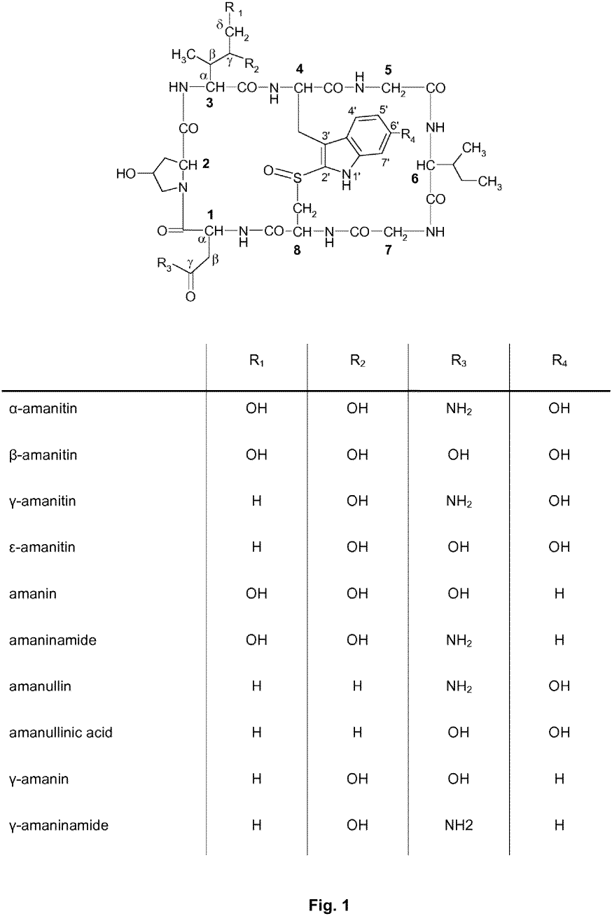

In the context of the present invention, the term “amanitins” particularly refers to bicyclic structure that are based on an aspartic acid or asparagine residue in position 1, a proline residue, particularly a hydroxyproline residue in position 2, an isoleucine, hydroxyisoleucine or dihydroxyisoleucine in position 3, a tryptophan or hydroxytryptophan residue in position 4, glycine residues in positions 5 and 7, an isoleucine residue in position 6, and a cysteine residue in position 8, particularly a derivative of cysteine that is oxidized to a sulfoxide or sulfone derivative (for the numbering and representative examples of amanitins, see FIG. 1), and furthermore includes all chemical derivatives thereof; further all semisynthetic analogues thereof; further all synthetic analogues thereof built from building blocks according to the master structure of the natural compounds (cyclic, 8 amino acids), further all synthetic or semisynthetic analogues containing non-hydroxylated amino acids instead of the hydroxylated amino acids, further all synthetic or semisynthetic analogues, in each case wherein any such derivative or analogue is functionally active by inhibiting mammalian RNA polymerase II.

In some embodiments of the inventive method as disclosed herein, the amatoxin is conjugated to the first and/or second antibody, whereby the amatoxin may be directly covalently linked to the antibody, or whereby the amatoxin may be covalently linked to the antibody via a linker, preferably, the amatoxin is covalently linked to the antibody via a linker.

According to a preferred embodiment, the first and/or second antibody drug conjugate (ADC) in the inventive method have the structure of

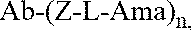

wherein Ab is an antibody or antigen-binding fragment thereof which is conjugated (covalently linked) to a linker (L), through a chemical moiety (Z), to an amatoxin moiety (Ama) and wherein n is from about 1, 2, 3, 4 to about 5, 6, 7, 8, or from about 5, 6, 7, to about 9, 10, or n is 1, 2, 3, 4, 5, 6, 7, 8, preferably n is between 1, 2 to 3, 3.5 or 4, more preferably n is about 2.

According to some embodiments, the first and/or second ADC for use according to the invention comprises an amatoxin (Ama) of Formula (A) (SEQ ID NO: 1)

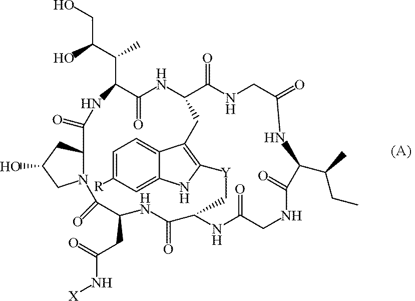

or a pharmaceutically acceptable salt, solvate, hydrate, tautomer, stereoisomer, diastereomer, or enantiomer thereof, wherein:
Y is —S—, —S(═O) —, or —SO₂—;
R is H, —OH, or —O—L—Z—Ab; and
X is H or —L—Z—Ab, wherein:
   L is a linker;
   Ab is an antibody as described herein or an antigen binding fragment thereof;
   Z is a chemical moiety formed by a coupling reaction between a first reactive substituent previously bound to L and a second reactive substituent previously present within the antibody, or antigen-binding fragment thereof;
provided that:
   if X is H then R is —O—L—Z—Ab, and
   if X is —L—Z—Ab then R is H or —OH.

As used herein, the term “antibody” shall refer to a protein consisting of one or more polypeptide chains encoded by immunoglobulin genes or fragments of immunoglobulin genes or cDNAs derived from the same. Said immunoglobulin genes include the light chain kappa, lambda and heavy chain alpha, delta, epsilon, gamma and mu constant region genes as well as any of the many different variable region genes.

The basic immunoglobulin (antibody) structural unit is usually a tetramer composed of two identical pairs of polypeptide chains, the light chains (L, having a molecular weight of about 25 kDa) and the heavy chains (H, having a molecular weight of about 50-70 kDa). Each heavy chain is comprised of a heavy chain variable region (abbreviated as VH or V_{H}) and a heavy chain constant region (abbreviated as CH or C_{H}). The heavy chain constant region is comprised of three domains, namely CH1, CH2 and CH3. Each light chain contains a light chain variable region (abbreviated as VL or V_{L}) and a light chain constant region (abbreviated as CL or C_{L}). The VH and VL regions can be further subdivided into regions of hypervariability, which are also called complementarity determining regions (CDR) interspersed with regions that are more conserved called framework regions (FR). Each VH and VL region is composed of three CDRs and four FRs arranged from the amino terminus to the carboxy terminus in the order of FR1, CDR1, FR2, CDR2, FR3, CDR3, FR4. The variable regions of the heavy and light chains form a binding domain that interacts with an antigen.

The CDRs are most important for binding of the antibody or the antigen binding portion thereof. The FRs can be replaced by other sequences, provided the three-dimensional structure which is required for binding of the antigen is retained. Structural changes of the construct most often lead to a loss of sufficient binding to the antigen.

The term “antigen binding portion” of the (monoclonal) antibody refers to one or more fragments of an antibody which retain the ability to specifically bind to the CD20 antigen in its native form. Examples of antigen binding portions of the antibody include a Fab fragment, a monovalent fragment consisting of the VL, VH, CL and CH1 domains, an F(ab′)₂ fragment, a bivalent fragment comprising two Fab fragments linked by a disulfid bridge at the hinge region, an Fd fragment consisting of the VH and CH1 domain, an Fv fragment consisting of the VL and VH domains of a single arm of an antibody, and a dAb fragment which consists of a VH domain and an isolated complementarity determining region (CDR).

The antibody, or antibody fragment or antibody derivative thereof, according to the present invention can be a monoclonal antibody. The antibody can be of the IgA, IgD, IgE, IgG or IgM isotype.

The term “monoclonal antibody” (“mAb”) as used herein refers to a preparation of antibody molecules of single specificity. A monoclonal antibody displays a single binding specificity and affinity for a particular epitope. Accordingly, the term “human monoclonal antibody” refers to an antibody displaying a single binding specificity which has variable and constant regions derived from or based on human germline immunoglobulin sequences or derived from completely synthetic sequences. The method of preparing the monoclonal antibody is not relevant for the binding specificity. Preferably, such antibody is selected from the group consisting of IgG, IgD, IgE, IgA and/or IgM, or a fragment or derivative thereof, more preferably such antibody is an IgG type antibody or fragment or derivative thereof.

As used herein, the term “fragment” shall refer to fragments of such antibody retaining target binding capacities, e.g., a CDR (complementarity determining region), a hypervariable region, a variable domain (Fv), an IgG heavy chain (consisting of VH, CH1, hinge, CH2 and CH3 regions), an IgG light chain (consisting of VL and CL regions), and/or a Fab and/or F(ab)₂.

As used herein, the term “antigen-binding fragment” shall refer to protein constructs being structurally different from, but still having some structural relationship to, the common antibody concept, e.g. a protein composed of a peptide scaffold and at least one of the CDRs of the original antibody it is derived from. Examples include e.g. scFv, Fab and/or F(ab)₂, as well as bi-, tri- or higher specific antibody constructs. All these items are explained below.

In some embodiments, the antibody of the first and/or second ADC for use according to the invention may e.g. be a Diabody, Camelid Antibody, Domain Antibody, a bivalent homodimer with two chains consisting of scFvs, IgAs (two IgG structures joined by a J chain and a secretory component), a shark antibody, or an antibody consisting of new world primate framework plus non-new world primate CDR, dimerised constructs comprising CH3+VL+VH, or other scaffold protein format comprising CDRs, and antibody conjugates (e.g., antibody, or fragments or derivatives thereof, linked to a drug, a toxin, a cytokine, an aptamer, a nucleic acid such as a desoxyribonucleic acid (DNA) or ribonucleic acid (RNA), a therapeutic polypeptide, a radioisotope or a label). Said scaffold protein formats may comprise, for example, antibody-like proteins such as ankyrin and affilin proteins and others, which are e.g. conjugated to at least 1, 2, 3, 4, 5, 6, 7, or 8 amatoxin molecules, or amatoxin-linker molecules as disclosed herein. For example, a corresponding ADC for use according to the invention as disclosed above may comprise from about 1, 2, 3 to about 4, 5, 6, 7, 8, or from about 4, 5, 6 to about 8, 9, 10 or from about 8 to about 10 amatoxin molecules, preferably linked to said ADC via a linker (L) as disclosed herein.

Antibodies generally are reflected by 10⁻⁵ to 10⁻¹¹ M or less dissociation constant in its cognate antigen (K_{D}), binds with high affinity. About 10⁻⁴ M greater than all the K_{D}, generally considered to refer to non-specific binding. The ADC or antibody-moiety of the respective ADC as used herein that “specifically binds” to an antigen is 10⁻⁷ M or less, preferably 10⁻⁸ M or less, 10⁻⁹ or less, 10⁻¹⁰ M or less, 10⁻¹¹ M or less, 10⁻¹² M or less. Specific binding of an antibody or antibody-moiety as comprised in the ADCs used in the inventive method may e.g. be determined by surface plasmon resonance analysis (Malmqvist M., “Surface plasmon resonance for detection and measurement of antibody-antigen affinity and kinetics.”, Curr Opin Immunol. 1993 Apr;5(2):282-6.).

The term “linker” (L) in the context of the present application refers to a molecule that increases the distance between two components, e.g. to alleviate steric interference between the target binding moiety and the amatoxin, which may otherwise decrease the ability of the amatoxin to interact with RNA polymerase II. The linker may serve another purpose as it may facilitate the release of the amatoxin specifically in the cell being targeted by the target binding moiety. It is preferred that the linker and preferably the bond between the linker and the amatoxin on one side and the bond between the linker and the target binding moiety or antibody on the other side is stable under the physiological conditions outside the cell, e.g. the blood, while it can be cleaved inside the cell, in particular inside the target cell, e.g. cancer cell. To provide this selective stability, the linker may comprise functionalities that are preferably pH-sensitive or protease sensitive. Altematively, the bond linking the linker to the target binding moiety may provide the selective stability. Preferably a linker has a length of at least 1, preferably of 1-30 atoms length (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 atoms), wherein one side of the linker has been reacted with the amatoxin and, the other side with a target-binding moiety. In the context of the present invention, a linker preferably is a C₁₋₃₀-alkyl, C₁₋₃₀-heteroalkyl, C₂₋₃₀-alkenyl, C₂₋₃₀-heteroalkenyl, C₂₋₃₀-alkynyl, C₂₋₃₀-heteroalkynyl, cycloalkyl, heterocycloalkyl, aryl, heteroaryl, aralkyl, or a heteroaralkyl group, optionally substituted. The linker may contain one or more structural elements such as amide, ester, ether, thioether, disulfide, hydrocarbon moieties and the like. The linker may also contain combinations of two or more of these structural elements. Each one of these structural elements may be present in the linker more than once, e.g. twice, three times, four times, five times, or six times. In some embodiments the linker may comprise a disulfide bond. It is understood that the linker has to be attached either in a single step or in two or more subsequent steps to the amatoxin and the target binding moiety. To that end the linker to be will carry two groups, preferably at a proximal and distal end, which can (i) form a covalent bond to a group, preferably an activated group on an amatoxin or the target binding-peptide or (ii) which is or can be activated to form a covalent bond with a group on an amatoxin. Accordingly, if the linker is present, it is preferred that chemical groups are at the distal and proximal end of the linker, which are the result of such a coupling reaction, e.g. an ester, an ether, a urethane, a peptide bond etc. In some embodiments, disclosed above, the presence of a “linker” is optional, i.e. the amatoxin may be directly linked to a residue of the antibody or antibody fragment.

The covalent attachment of the antibody and the amatoxin according to the invention requires the linker to have two reactive functional groups, i.e. bivalency in a reactive sense. Bivalent linker reagents which are useful to attach two or more functional or biologically active moieties, such as peptides, nucleic acids, drugs, toxins, antibodies, haptens, and reporter groups are known, and methods have been described their resulting conjugates (Hermanson, G. T. (1996) Bioconjugate Techniques; Academic Press: New York, p. 234-242).

Accordingly, present linkers have two reactive termini, one for conjugation to an antibody and the other for conjugation to a amatoxin. The antibody conjugation reactive terminus of the linker (reactive moiety, defined herein as Z′) is typically a chemical moiety that is capable of conjugation to the antibody through, e.g., a cysteine thiol or lysine amine group on the antibody, and so is typically a thiol-reactive group such as a Michael acceptor (as in maleimide), a leaving group, such as a chloro, bromo, iodo, or an R-sulfanyl group, or an amine-reactive group such as a carboxyl group.

The amatoxin conjugation reactive terminus of the linker is typically a chemical moiety that is capable of conjugation to the amatoxin through formation of a bond with a reactive substituent within the amatoxin molecule. Non-limiting examples include, for example, formation of an amide bond with a basic amine or carboxyl group on the amatoxin, via a carboxyl or basic amine group on the linker, respectively, or formation of an ether or the like, via alkylation of an OH group on the amatoxin via e.g., a leaving group on the linker.

When the term “linker” (L) is used in describing the linker in conjugated form, one or both of the reactive termini will be absent (such as reactive moiety Z′, having been converted to chemical moiety Z, as described herein below) or incomplete (such as being only the carbonyl of the carboxylic acid) because of the formation of the bonds between the linker and/or the amatoxin, and between the linker and/or the antibody or antigen-binding fragment thereof.

A variety of linkers can be used to conjugate the antibodies, antigen-binding fragments, and ligands described to a cytotoxic molecule. Generally, linkers suitable for the present disclosure should be stable in circulation, but allow for release of the amatoxin within or in close proximity to the target cells. Linkers suitable for the present disclosure may be broadly categorized as non-cleavable or cleavable, each of which is further described herein below.

The linkers useful for the present amatoxin ADCs are preferably stable extracellularly, prevent aggregation of ADC molecules and keep the ADC freely soluble in aqueous media and in a monomeric state. Before transport or delivery into a cell, the ADC is preferably stable and remains intact (i.e. the antibody remains linked to the amatoxin outside the target cell and may be cleaved at some efficacious rate inside the cell).

In some embodiments, the first and/or second ADC for use according to the inventive method comprise a cleavable or a non-cleavable linker. The term “non-cleavable linker” refers to linkers which comprise chemical bonds that are resistant to degradation (e.g., proteolysis). Generally, non-cleavable linkers require proteolytic degradation inside the target cell, and exhibit high extracellular stability. Non-cleavable linkers suitable for use herein further may include one or more groups selected from a bond, —(C═O)—, C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, and combinations thereof, each of which may be optionally substituted, and/or may include one or more heteroatoms (e.g., S, N, or O) in place of one or more carbon atoms. Non-limiting examples of such groups include (CH₂)ₚ, (C═O)(CH₂)ₚ, and polyethyleneglycol (PEG; (CH₂CH₂O)ₚ), units, wherein p is an integer from 1-6, independently selected for each occasion.

In some embodiments, the non-cleavable linker L comprises one or more of a bond, —(C═O)—, a —C(O)NH— group, an —OC(O)NH— group, C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, a —(CH₂CH₂O)ₚ— group where p is an integer from 1-6, or a solubility enhancing group; wherein each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene may optionally be substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene of the non-cleavable linker as disclosed herein may optionally be interrupted by one or more heteroatoms selected from O, S and N.

In some embodiments, each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene of the non-cleavable linker as disclosed herein may optionally be interrupted by one or more heteroatoms selected from O, S and N and may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, the non-cleavable linker according to the invention comprises a —(CH₂)ₙ—unit, where n is an integer from 2 to 6, 7, 8, 9, 10, 11, 12, preferably n is an integer from 2 to 6, 7, 8, more preferably, n is an integer from about 2 to 6, e.g. 2, 3, 4, 5, 6, even more preferred, the non-cleavable linker comprises a —(CH₂)ₙ— where n is 6. In some embodiments, the non-cleavable linker is —(CH₂)ₙ— where n is 6, represented by the formula:

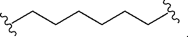

In some embodiments, the non-cleavable linkers of the first and/or second ADC for use according to the invention as disclosed herein further comprise a thiol-reactive group prior to the conjugation to the antibody. The thiol-reactive group of said non-cleavable linkers as disclosed above may e.g. be selected from bromo acetamide, iodo acetamide, methylsulfonylbenzothiazole, 4,6-dichloro-1,3,5-triazin-2-ylamino group methyl-sulfonyl phenyltetrazole or methylsulfonyl phenyloxadiazole, pyridine-2-thiol, 5- nitropyridine-2-thiol, methanethiosulfonate, or a maleimide, preferably the thiol reactive group is a maleimide (maleimidyl moiety). For example, the non-cleavable linker comprising said maleimide may have the following structure prior to conjugation to a respective antibody, whereby the wavy line at the linker terminus indicates the point of attachment to the amatoxin (Ama):

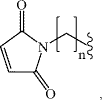

wherein n is an integer from 2-12, e.g. n is 2, 3, 4, 5, 6, 7, 8, 9, or 10, preferably wherein n is an integer from 2-6, e.g. n is 1, 2, 3, 4, 5, or 6, more preferably, n is 6.

Following conjugation to a reactive sulfhydryl on an antibody or reactive disulfide bonds, e.g. a naturally occurring cysteine residues of the antibody, or e.g. interchain disulfide bonds, or engineered cysteine residues, the maleimidyl moiety of e.g. cleavable or non-cleavable linker as disclosed herein comprise the structure:

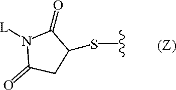

whereby the wavy line represents the attachment site of a cleavable or non-cleavable linker (L) as disclosed herein and the sulfur atom is part of a reactive cysteine of the antibody.

According to preferred embodiments, the first and/or second ADC for use according to the invention comprise a cleavable or non-cleavable linker as disclosed herein and which further comprise a thiol-reactive group as disclosed herein may be coupled to a naturally occurring sulfhydryl moiety, or disulfide bond in the antibody, or said cleavable or non-cleavable linker of the first and/or second ADC for use according to the invention comprises a thiol-reactive group which may be coupled to a sulfhydryl moiety which has been introduced into the antibody by genetic engineering as described in e.g. Nat Biotechnol. 2008 Aug;26(8):925-32, or WO2006/034488 A2. Preferably, the cleavable or non-cleavable linker of said first and/or second ADC for use as disclosed herein comprise a thiol-reactive group are coupled to sulfhydryl moieties that have been introduced into the Fc region of the respective antibody of the said first and/or second ADC for use according the invention by genetic engineering such as e.g. D265C (according to EU numbering, Edelman, G.M. et al., Proc. Natl. Acad. USA, 63, 78-85 (1969).

According to some embodiments, the linker of the first and/or second ADC for use according to the inventive method as disclosed herein is connected to the at least one amatoxin via (i) the γ C-atom of amatoxin amino acid 1, or (ii) the δ C-atom of amatoxin amino acid 3, or (iii) the 6′-C-atom of amatoxin amino acid 4.

In one embodiment, said first and/or second ADC for use according to the invention comprises a conjugate as described herein, which comprises an amatoxin comprising (i) an amino acid 4 with a 6′-deoxy position and (ii) an amino acid 8 with an S-deoxy position.

In some embodiments, the linker of the first and/or second ADC for use in the inventive methodis a cleavable linker. The term “cleavable linker” is understood as comprising at least one cleavage site. As used herein, the term “cleavage site” shall refer to a moiety that is susceptible to specific cleavage at a defined position under particular conditions. Said conditions are, e.g., specific enzymes or a reductive environment in specific body or cell compartments.

According to some embodiments, the cleavage site of said linker is an enzymatically cleavable moiety comprising two or more amino acids. Preferably, said enzymatically cleavable moiety comprises a valine-alanine (Val-Ala), valine-citrulline (Val-Cit), valine-lysine (Val-Lys), valine-arginine (Val-Arg) dipeptide, a phenylalanine-lysine-glycine-proline-leucin-glycine (Phe Lys Gly Pro Leu Gly) or alanine-alanine-proline-valine (Ala Ala Pro Val) peptide, or a β-glucuronide or β-galactoside.

According to some embodiments, said cleavage site can be cleavable by at least one protease selected from the group consisting of cysteine protease, metalloprotease, serine protease, threonine protease, and aspartic protease.

Cysteine proteases, also known as thiol proteases, are proteases that share a common catalytic mechanism that involves a nucleophilic cysteine thiol in a catalytic triad or dyad.

Metalloproteases are proteases whose catalytic mechanism involves a metal. Most metalloproteases require zinc, but some use cobalt. The metal ion is coordinated to the protein via three ligands. The ligands co-ordinating the metal ion can vary with histidine, glutamate, aspartate, lysine, and arginine. The fourth coordination position is taken up by a labile water molecule.

Serine proteases are enzymes that cleave peptide bonds in proteins; serine serves as the nucleophilic amino acid at the enzyme’s active site. Serine proteases fall into two broad categories based on their structure: chymotrypsin-like (trypsin-like) or subtilisin-like.

Threonine proteases are a family of proteolytic enzymes harbouring a threonine (Thr) residue within the active site. The prototype members of this class of enzymes are the catalytic subunits of the proteasome, however, the acyltransferases convergently evolved the same active site geometry and mechanism.

Aspartic proteases are a catalytic type of protease enzymes that use an activated water molecule bound to one or more aspartate residues for catalysis of their peptide substrates. In general, they have two highly conserved aspartates in the active site and are optimally active at acidic pH. Nearly all known aspartyl proteases are inhibited by pepstatin.

In some embodiments of the present invention, the cleavable site is cleavable by at least one agent selected from the group consisting of Cathepsin A or B, matrix metalloproteinases (MMPs), elastases, β-glucuronidase and β-galactosidase.

In some embodiments of the present invention, the cleavage site is a disulfide bond and specific cleavage is conducted by a reductive environment, e.g., an intracellular reductive environment, such as, e.g., acidic pH conditions.

Suitable cleavable linkers include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference as it pertains to linkers suitable for covalent conjugation). Suitable cleavable linkers may include, for example, chemical moieties such as a hydrazine, a disulfide, a thioether or a dipeptide.

Linkers hydrolyzable under acidic conditions include, for example, hydrazones, semicarbazones, thiosemicarbazones, cis-aconitic amides, orthoesters, acetals, ketals, or the like. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661, the disclosure of each of which is incorporated herein by reference in its entirety as it pertains to linkers suitable for covalent conjugation. Such linkers are relatively stable under neutral pH conditions, such as those in the blood, but are unstable at below pH 5.5 or 5.0, the approximate pH of the lysosome.

Linkers cleavable under reducing conditions include, for example, a disulfide. A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alphamethyl-alpha-(2-pyridyl-dithio)toluene), SPDB and SMPT (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935, the disclosure of each of which is incorporated herein by reference in its entirety as it pertains to linkers suitable for covalent conjugation.

Linkers susceptible to enzymatic hydrolysis can be, e.g., a peptide-containing linker that is cleaved by an intracellular peptidase or protease enzyme, including, but not limited to, a lysosomal or endosomal protease. One advantage of using intracellular proteolytic release of the therapeutic agent is that the agent is typically attenuated when conjugated and the serum stabilities of the conjugates are typically high. In some embodiments, the peptidyl linker is at least two amino acids long or at least three amino acids long. Exemplary amino acid linkers include a dipeptide, a tripeptide, a tetrapeptide or a pentapeptide. Examples of suitable peptides include those containing amino acids such as Valine, Alanine, Citrulline (Cit), Phenylalanine, Lysine, Leucine, and Glycine. Amino acid residues which comprise an amino acid linker component include those occurring naturally, as well as minor amino acids and non-naturally occurring amino acid analogs, such as citrulline. Exemplary dipeptides include valine-citrulline (vc or val-cit) and alanine-phenylalanine (af or ala-phe). Exemplary tripeptides include glycine-valine-citrulline (gly-val-cit) and glycine-glycine-glycine (gly-gly-gly). In some embodiments, the linker includes a dipeptide such as Val-Cit, Ala-Val, or Phe-Lys, Val-Lys, Ala-Lys, Phe-Cit, Leu-Cit, Ile-Cit, Phe-Arg, or Trp-Cit. Linkers containing dipeptides such as Val-Cit or Phe-Lys are disclosed in, for example, U.S. Pat. No. 6,214,345, the disclosure of which is incorporated herein by reference in its entirety as it pertains to linkers suitable for covalent conjugation. In some embodiments, the linker comprises a dipeptide selected from Val-Ala and Val-Cit.

Linkers suitable for conjugating the antibody moieties of the first and/or second ADC of the invention or antigen-binding fragments to a cytotoxic molecule include those capable of releasing an amatoxin by a 1,6-elimination process. Chemical moieties capable of this elimination process include the p-aminobenzyl (PAB) group, 6-maleimidohexanoic acid, pH-sensitive carbonates, and other reagents as described in Jain et al., Pharm. Res. 32:3526-3540, 2015, the disclosure of which is incorporated herein by reference in its entirety as it pertains to linkers suitable for covalent conjugation.

In some embodiments, the cleavable linker as disclosed herein linker includes a “self-immolative” group such as the afore-mentioned PAB or PABC (para-aminobenzyloxycarbonyl), which are disclosed in, for example, Carl et al., J. Med. Chem. (1981) 24:479-480; Chakravarty et al (1983) J. Med. Chem. 26:638-644; US 6214345; US20030130189; US20030096743; US6759509; US20040052793; US6218519; US6835807; US6268488; US20040018194; W098/13059; US20040052793; US6677435; US5621002; US20040121940; W02004/032828). Other such chemical moieties capable of this process (“self-immolative linkers”) include methylene carbamates and heteroaryl groups such as aminothiazoles, aminoimidazoles, aminopyrimidines, and the like. Linkers containing such heterocyclic self-immolative groups are disclosed in, for example, U.S. Pat. Publication Nos. 20160303254 and 20150079114, and U.S. Pat. No. 7,754,681; Hay et al. (1999) Bioorg. Med. Chem. Lett. 9:2237; US 2005/0256030; de Groot et al (2001) J. Org. Chem. 66:8815-8830; and US 7223837. In some embodiments, a dipeptide is used in combination with a self-immolative linker.

In some embodiments, the linker L comprises one or more of a hydrazine, a disulfide, a thioether, an amino acid, a peptide consisting of up to 10 amino acids, a p-aminobenzyl (PAB) group, a heterocyclic self-immolative group, C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, heteroaryl, a —(C═O)— group, a —C(O)NH— group, an —OC(O)NH— group, a —(CH₂CH₂O)ₚ— group where p is an integer from 1-6, or a solubility enhancing group; wherein each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may optionally be interrupted by one or more heteroatoms selected from O, S and N.

In some embodiments, each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may optionally be interrupted by one or more heteroatoms selected from O, S and N and may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, the linker includes a p-aminobenzyl group (PAB). In one embodiment, the p-aminobenzyl group is disposed between the cytotoxic drug and a protease cleavage site in the linker. In one embodiment, the p-aminobenzyl group is part of a p-aminobenzyloxycarbonyl unit. In one embodiment, the p-aminobenzyl group is part of a p-aminobenzylamido unit.

According to preferred embodiments, the cleavble linker of the first and/or second ADC as disclosed herein comprises a dipeptide selected from the group consisting of Phe-Lys, Val-Lys, Phe-Ala, Phe-Cit, Val-Ala, Val-Cit, and Val-Arg.

According to particularly preferred embodiments, the linker comprises one or more of PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In particularly preferred embodiments, the cleavable linker according to the invention comprises a dipeptide selected from Phe-Lys, Val-Lys, Phe-Ala, Val-Ala, Phe-Cit and Val-Cit, particularly wherein the cleavable linker further comprises a p-aminobenzyl (PAB) spacer between the dipeptides and the amatoxin:

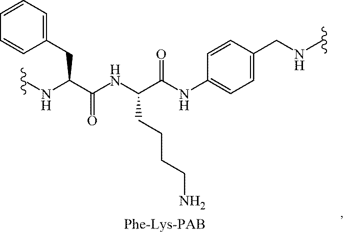

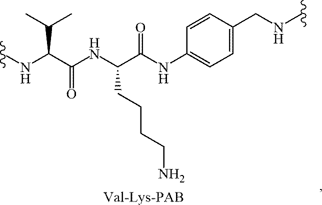

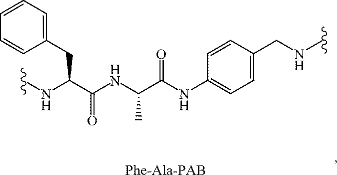

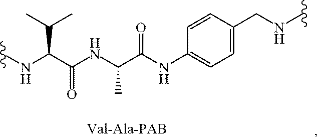

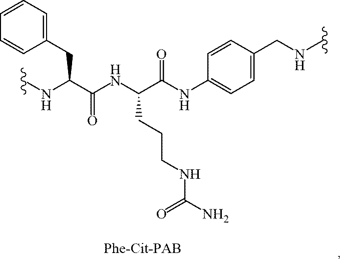

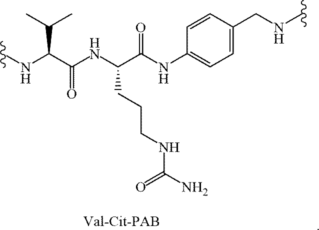

Accordingly, the first and/or second ADC of the inventive method as disclosed herein can comprise a cleavable linker which comprises any one of the dipeptides-PAB moieties Phe-Lys-PAB, Val-LysPAB, Phe-Ala-PAB, Val-Ala-PAB, Phe-Cit-PAB, or Val-Cit-PAB as disclosed above, peferably, the cleavable linker of the first and/or second ADC of the inventive method comprises the dipeptide-PAB moiety Val-Ala-PAB:

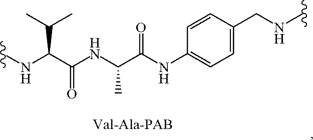

whereby the PAB moiety is linked to the amatoxin.

According to some embodiments, said cleavable linkers as disclosed herein comprise prior to conjugation to the antibody a thiol-reactive group, selected from bromo acetamide, iodo acetamide, methylsulfonylbenzothiazole, 4,6-dichloro-1,3,5-triazin-2-ylamino group methyl-sulfonyl phenyltetrazole or methylsulfonyl phenyloxadiazole, pyridine-2-thiol, 5- nitropyridine-2-thiol, methanethiosulfonate, or a maleimide.

According to a preferred embodiment the thiol reactive group is a maleimide (maleimidyl moiety) as depicted below:

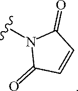

Linkers (e.g. cleavable and/or non-cleavable linkers) which comprise said thiol-reactive groups are particularly useful for covalent coupling of linker-amatoxin conjugates as disclosed herein to antibodies comprising reactive thiols, such as e.g. cysteine-engineered antibodies comprising at least one reactive cysteine residue for coupling as disclosed in Junutula et al. (2008) Nat Biotechnology Vol. 26: 925-932.

According to a preferred embodiment, the cleavable linker of the invention comprises the structure (i) prior to coupling, or (ii) following the coupling to an antibody (Ab) as disclosed herein:, whereby (ama) indicates the attachment site to the amatoxin and Ab to the antibody:

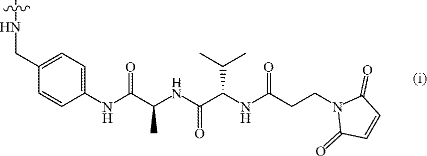

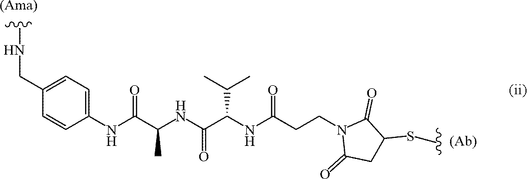

According to a particularly preferred embodiment, the non-cleavable linker of the first and/or second ADC for use in the inventive method comprises the structure prior to coupling (“ncl-1”) to the antibody of the first and/or second ADC for use in the inventive method:

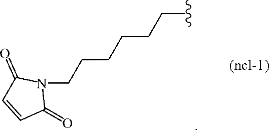

According to a particularly preferred embodiment, the non-cleavable linker of the first and/or second ADC of the invention comprises the structure (Z), wherein (ama) indicates the side of the linker that is connected to the amatoxin and Ab the side which is connected to the antibody, whereby the sulfur atom is part of the antibody, e.g. a genetically introduced cysteine residue, or a naturally occurring sulfur atom such as those comprised in interchain disulfide bonds:

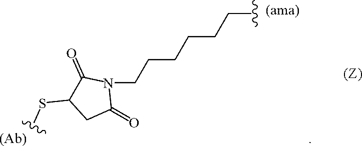

In some embodiments, the first and/or second ADC for use in the inventive method comprises an amatoxin (Ama) according to Formula (A1) (SEQ ID NO: 1):

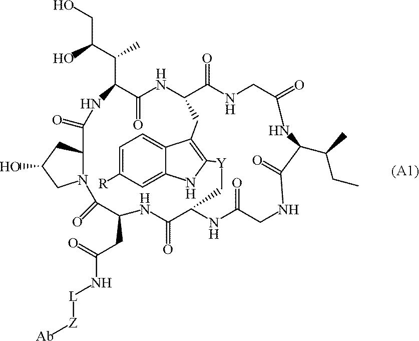

or a pharmaceutically acceptable salt, solvate, hydrate, tautomer, stereoisomer, diastereomer, or enantiomer thereof, wherein R is H or OH, and wherein Y is —S—, —S(═O) —, L is a cleavable or non-cleavable linker as disclosed herein and Z is as disclosed herein.

In some embodiments, the the first and/or second ADC for use in the inventive method of Formula (A) is of the Formula (A2) (SEQ ID NO:1):

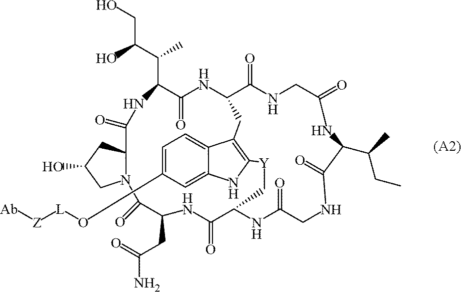

or a pharmaceutically acceptable salt, solvate, hydrate, tautomer, stereoisomer, diastereomer, or enantiomer thereof, wherein Y, L, Z′ and Ab are as defined above, preferably, Y is —S—, or —(S═O)—.

In some embodiments, the the first and/or second ADC for use according to the invention ADC of Formula (A) is of the Formula (A2) (SEQ ID NO:2):

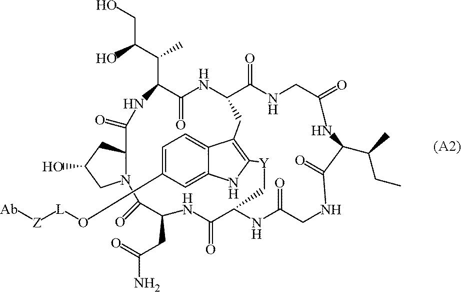

or a pharmaceutically acceptable salt, solvate, hydrate, tautomer, stereoisomer, diastereomer, or enantiomer thereof, wherein L, Z and Ab are as defined above and Y is —S—.

In some embodiments, the amatoxin-linker conjugate Ama-L of the first and/or second ADC for use in the inventive method is selected from the group of compounds comprising of formulae (I) to (XI):

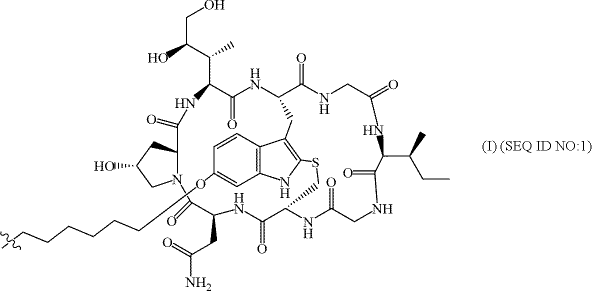

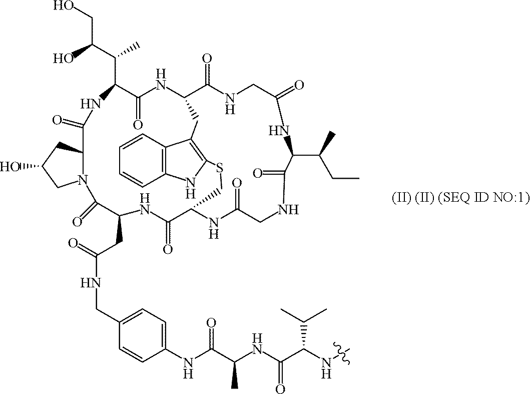

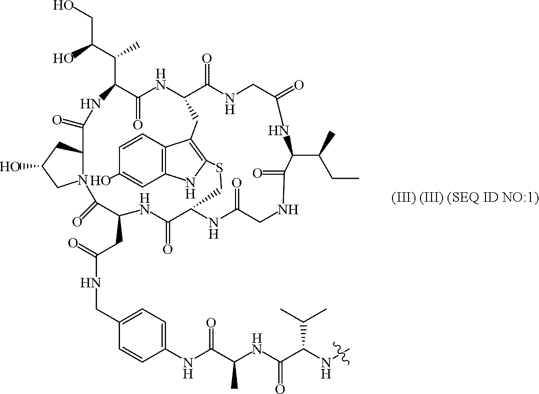

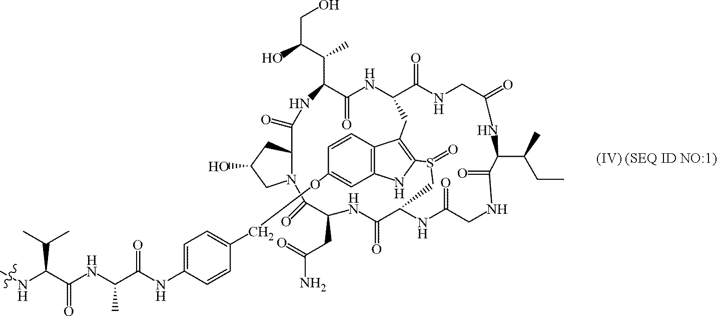

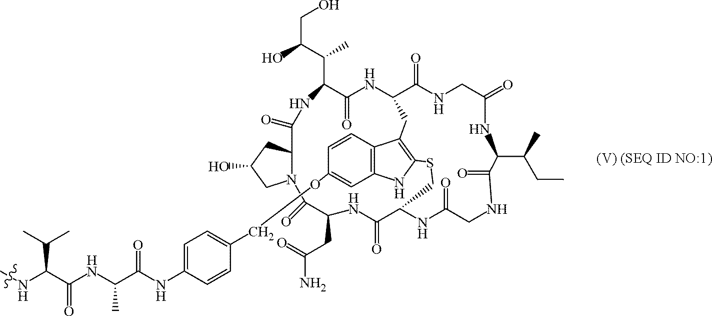

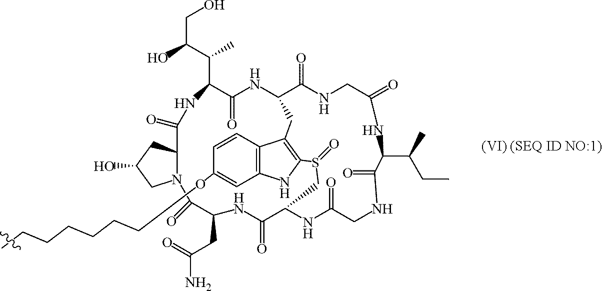

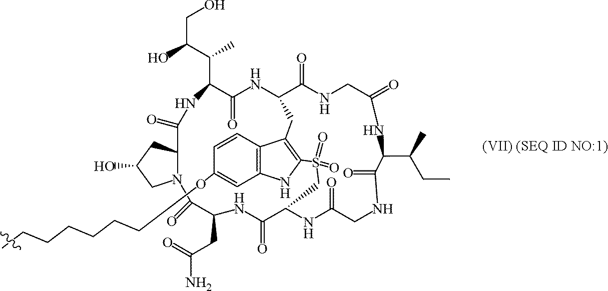

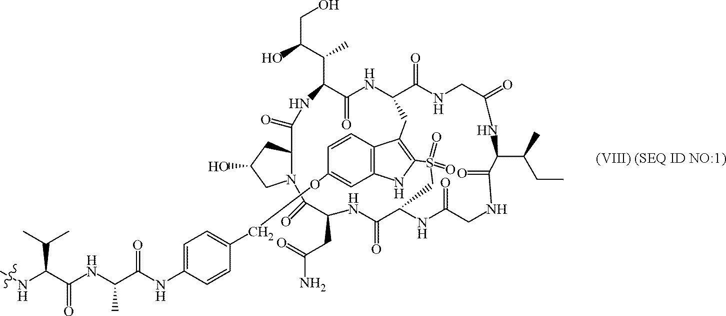

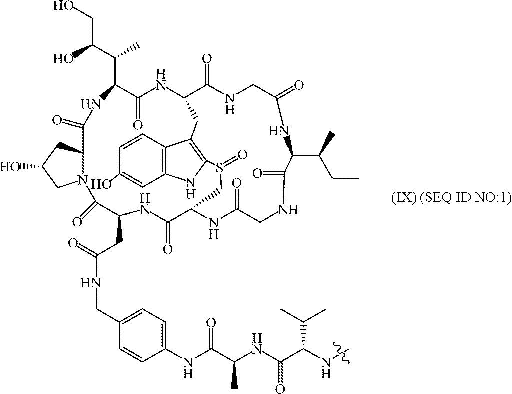

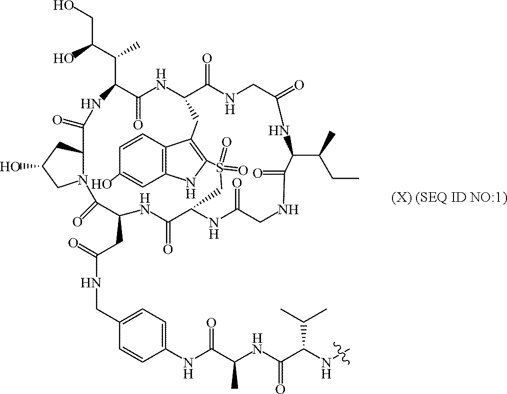

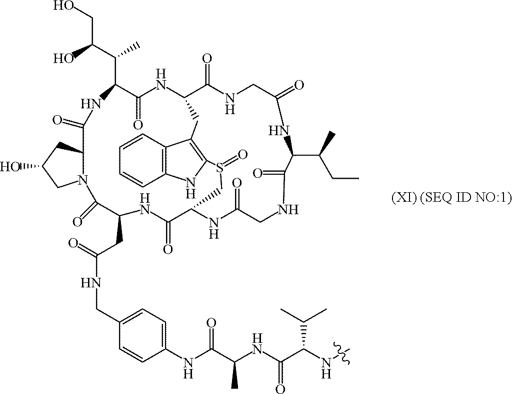

For example, the amatoxin-linker conjugate Ama-L of the first ADC for use in the inventive method is selected from the compounds (I) - (XI) above and the second ADC for use in the inventive method is selected from the compounds (I) - (XI) above. In one example, the amatoxin-linker conjugate Ama-L of the first ADC for use in the inventive method is selected from the compounds (I), (II), or (III), and the second ADC for use in the inventive method is selected from the compounds (I), (II), or (III).

According to some embodiments, the amatoxin-linker conjugate Ama-L of the first ADC for use in the inventive method is the same as the amatoxin-linker conjugate Ama-L of the second ADC for use in the inventive method, both of which are selected from any of the compounds (I) - (XII) above. For example, the amatoxin-linker conjugate Ama-L of the first and second ADC for use in the inventive method are selected from compounds (I), (II), or (III).

According to some embodiments, the amatoxin-linker conjugate Ama-L of the first ADC for use according to the inventive method is the same as the amatoxin-linker conjugate Ama-L of the second ADC for use according to the inventive method, both of which are selected from compounds (I), (II), or (III) as disclosed above.

In some embodiments, the first ADC for use in the inventive method as disclosed herein can e.g. comprise a different amatoxin-linker conjugate AMA-L than the second ADC for use in the inventive method.

For example, the first ADC for use in the inventive method may comprise one of the AMA-L conjugates or compounds of the left column, while the second ADC for use in the inventive method comprises an AMA-L conjugate or compound selected from the right column:

| | |
|---|---|
| AMA-L comprised in first ADC | AMA-L comprised in second ADC |
| (I) | (II), (III), (IV), (V), (VI), (VII), (VIII), (IX), (X), (XI) |
| (II) | (I), (III), (IV), (V), (VI), (VII), (VIII), (IX), (X), (XI) |
| (III) | (I), (II), (IV), (V), (VI), (VII), (VIII), (IX), (X), (XI) |
| (IV) | (I), (II), (III), (V), (VI), (VII), (VIII), (IX), (X), (XI) |
| (V) | (I), (II), (III), (IV), (VI), (VII), (VIII), (IX), (X), (XI) |
| (VI) | (I), (II), (III), (IV), (V), (VII), (VIII), (IX), (X), (XI) |
| (VII) | (I), (II), (III), (IV), (V), (VI), (VIII), (IX), (X), (XI) |
| (VIII) | (I), (II), (III), (IV), (V), (VI), (VII), (IX), (X), (XI) |
| (IX) | (I), (II), (III), (IV), (V), (VI), (VII), (VIII), (X), (XI) |
| (X) | (I), (II), (III), (IV), (V), (VI), (VII), (VIII), (IX), (XI) |
| (XI) | (I), (II), (III), (IV), (V), (VI), (VII), (VIII), (IX), (X) |

According to preferred embodiments, the first ADC for use in the inventive method comprises the amatoxin-linker conjugate Ama-L AMA-L according to formula (I) and the second ADC for use in the inventive method comprises an amatoxin-linker conjugate Ama-L AMA-L according to any of formulae (I), (II), (III), or (IX).

According to preferred embodiments, the first ADC for use in the inventive method comprises the amatoxin-linker conjugate Ama-L AMA-L according to formula (II) and the second ADC for use in the inventive method comprises an amatoxin-linker conjugate Ama-L AMA-L according to any of formulae (I), (II), (III), or (IX).

According to preferred embodiments, the first ADC for use in the inventive method comprises the amatoxin-linker conjugate Ama-L AMA-L according to formula (III) and the second ADC for use in the inventive method comprises an amatoxin-linker conjugate Ama-L according to any of formulae (I), (II), (III), or (IX).

According to more preferred embodiments, the first ADC for use in the inventive method comprises the amatoxin-linker conjugate Ama-L according to formula (I) and the second ADC for use in the inventive method comprises an amatoxin-linker conjugate Ama-L AMA-L according one of formulae (I), or (II).

According to more preferred embodiments, the first ADC for use in the inventive method comprises the amatoxin-linker conjugate Ama-L according to formula (II) and the second ADC for use in the inventive method comprises an amatoxin-linker conjugate Ama-L according one of formulae (I), or (II).

According to more preferred embodiments, the first ADC for use in the inventive method comprises the amatoxin-linker conjugate Ama-L according to formula (III) and the second ADC for use in the inventive method comprises an amatoxin-linker conjugate Ama-L according one of formulae (I), or (II).

According to a preferred embodiments, the first and/or second ADC for use in the inventive method as disclosed herein comprises a compound according to any one of formulae (XII) to (XXII) as disclosed hereinbelow, whereby “antibody” refers to the antibody moiety of the first and/or second ADC of the inventive method:

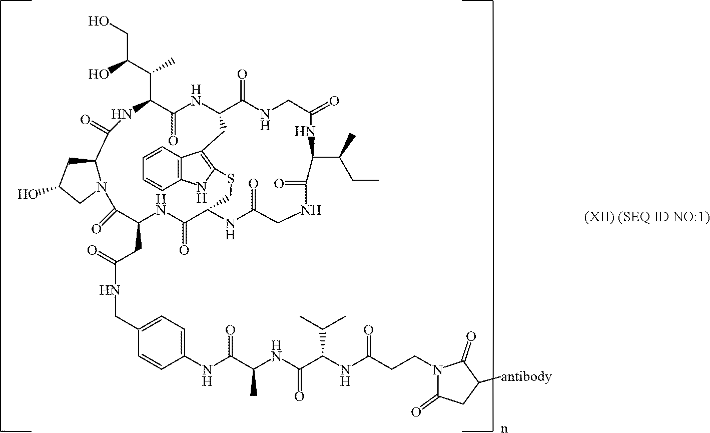

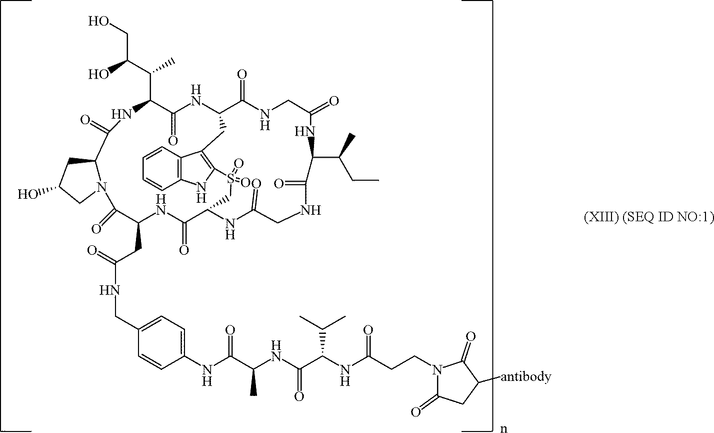

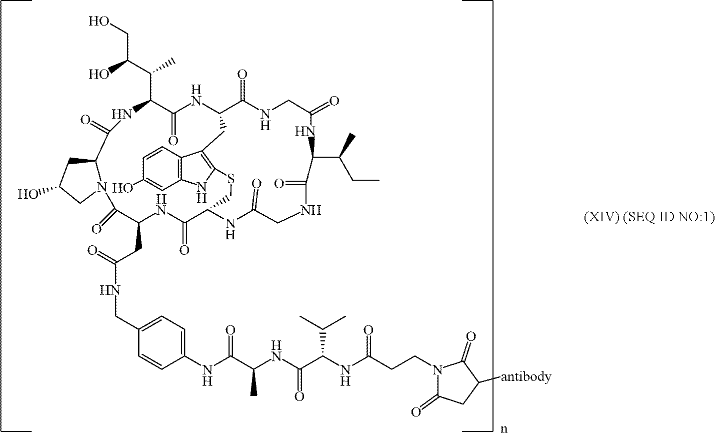

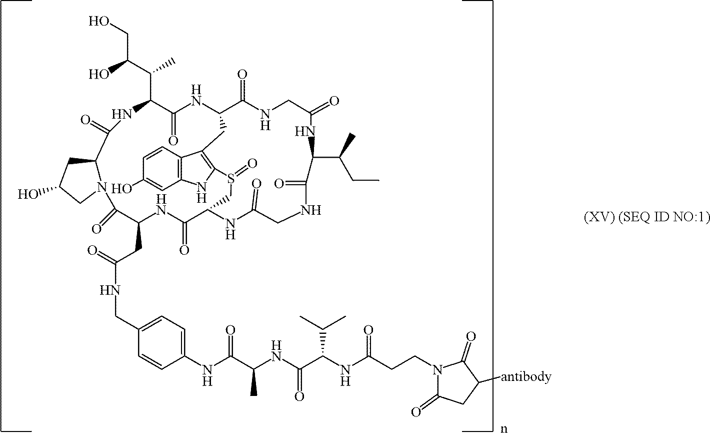

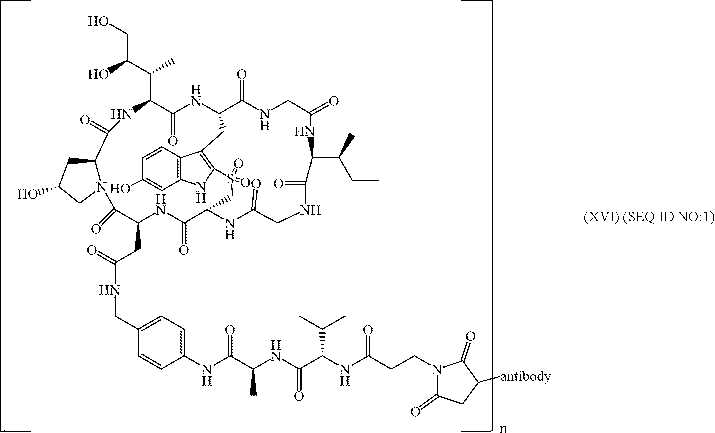

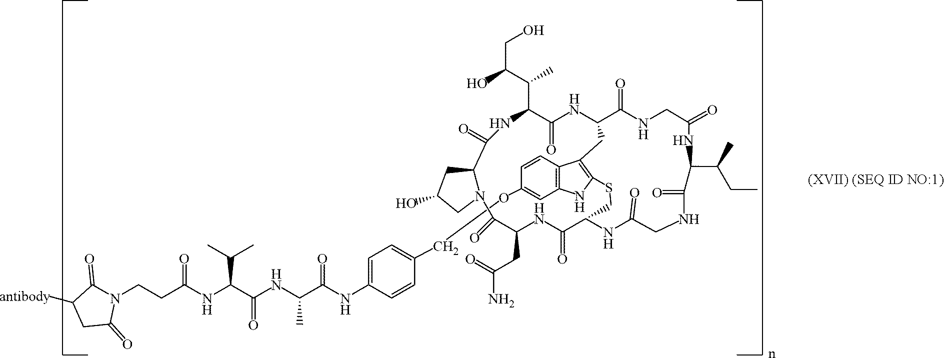

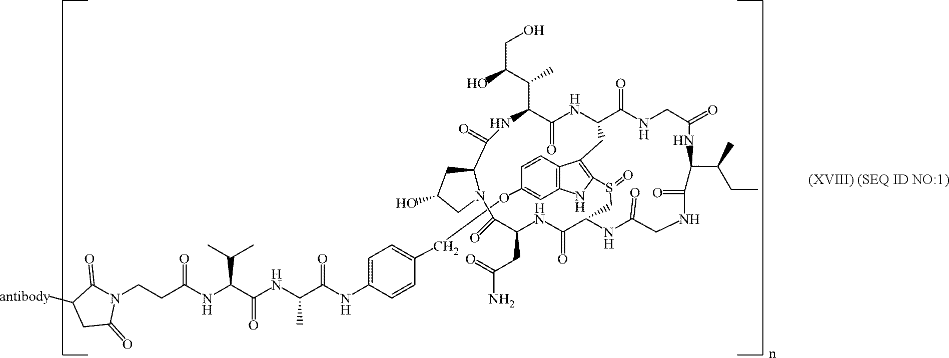

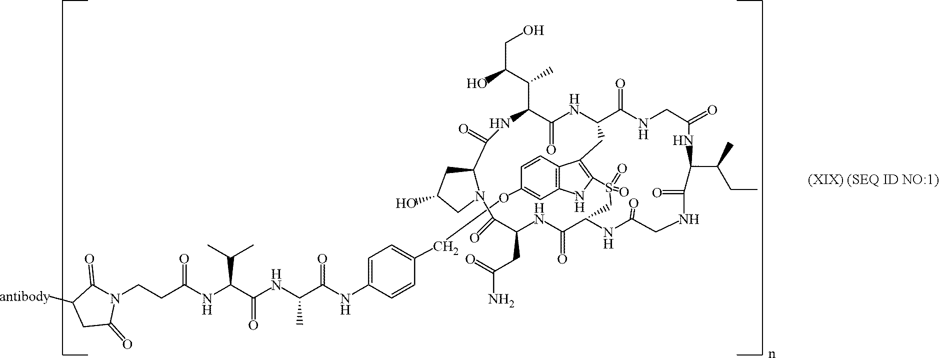

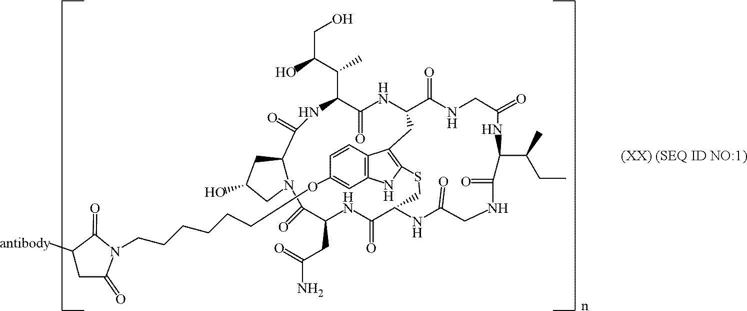

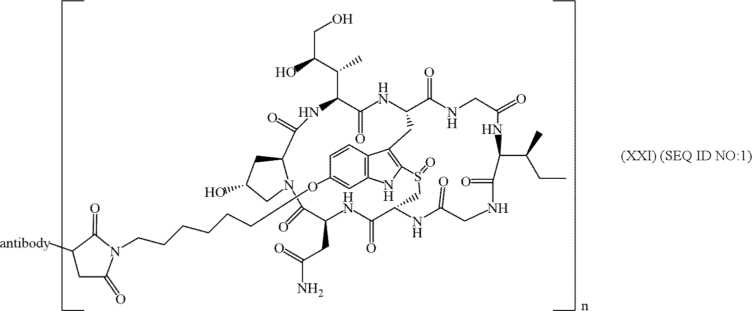

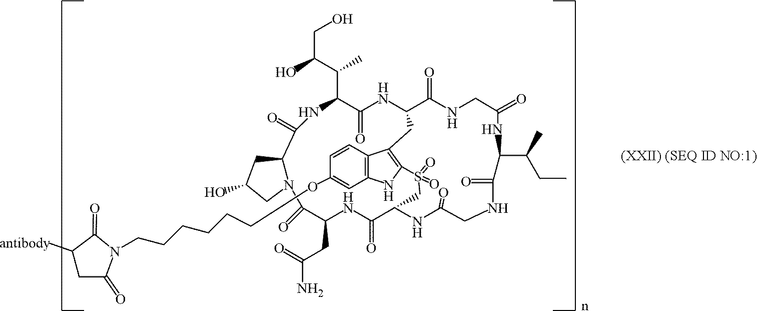

n is from about 1, 2, 3, 4 to about 5, 6, 7, 8, or from about 5, 6, 7, to about 9, 10, or n is 1, 2, 3, 4, 5, 6, 7, 8, preferably n is between 1 to 4, e.g. 1, 2, 3, 4, more preferably n is from about 1.5, 2.5 to about 3, 3.5 more preferably n is about 1.5 to about 2.5, or preferably n is about 2. The term “antibody” as used in any one of formulae (XII) to (XXII) shall indicate the conjugation to a suitable antibody, e.g. an antibody which binds to the desired antigen (cell surface antigen, tumor-asssociated antigen, or tumor specific antigen). It is to be understood that the inventive method as disclosed herein is applicable to any amatoxin-based ADC which comprises an amatoxin linker moiety according to any one of formulae (I) to (XI) having the corresponding structures as depicted in formulae (XII) to (XXII) subsequent to conjugation.

According to one embodiment, the first ADC for use in the inventive method is selected from an ADC according to any of formulae (XII) to (XXII) and a second ADC for use in the inventive method is selected from an ADC according to any of formulae (XII) to (XXII). For example, the first and second ADC for use in the inventive method may comprise the following combinations

| | |
|---|---|
| first ADC | second ADC |
| (XII) | (XII), (XIII), (XIV), (XV), (XVI), (XVII), (XVIII), (XIX), (XX), (XXI), (XXII) |
| (XIII) | (XII), (XIII), (XIV), (XV), (XVI), (XVII), (XVIII), (XIX), (XX), (XXI), (XXII) |
| (XIV) | (XII), (XIII), (XIV), (XV), (XVI), (XVII), (XVIII), (XIX), (XX), (XXI), (XXII) |
| (XV) | (XII), (XIII), (XIV), (XV), (XVI), (XVII), (XVIII), (XIX), (XX), (XXI), (XXII) |
| (XVI) | (XII), (XIII), (XIV), (XV), (XVI), (XVII), (XVIII), (XIX), (XX), (XXI), (XXII) |
| (XVII) | (XII), (XIII), (XIV), (XV), (XVI), (XVII), (XVIII), (XIX), (XX), (XXI), (XXII) |
| (XVIII) | (XII), (XIII), (XIV), (XV), (XVI), (XVII), (XVIII), (XIX), (XX), (XXI), (XXII) |
| (XIX) | (XII), (XIII), (XIV), (XV), (XVI), (XVII), (XVIII), (XIX), (XX), (XXI), (XXII) |
| (XX) | (XII), (XIII), (XIV), (XV), (XVI), (XVII), (XVIII), (XIX), (XX), (XXI), (XXII) |
| (XXI) | (XII), (XIII), (XIV), (XV), (XVI), (XVII), (XVIII), (XIX), (XX), (XXI), (XXII) |
| (XXII) | (XII), (XIII), (XIV), (XV), (XVI), (XVII), (XVIII), (XIX), (XX), (XXI), (XXII) |

According to some embodiments, the first and second ADC for use in the inventive method may be the same ADC, or may be different ADCs, however, it is preferred that the first and second ADC for use in the inventive method are the same ADC.

According to preferred embodiments, the first ADC for use in the inventive method comprises an ADC according to formula (XII) and a second ADC for use in the inventive method according to one of formulae (XII), (XIV), (XV), or (XX).

According to preferred embodiments, the first ADC for use in the inventive method comprises an ADC according to formula (XIV) and a second ADC for use in the inventive method according to one of formulae (XII), (XIV), (XV), or (XX).

According to preferred embodiments, the first ADC for use in the inventive method comprises an ADC according to formula (XV) and a second ADC for use in the inventive method according to one of formulae (XII), (XIV), (XV), or (XX).

According to preferred embodiments, the first ADC for use in the inventive method comprises an ADC according to formula (XX) and a second ADC for use in the inventive method according to one of formulae (XII), (XIV), (XV), or (XX).

According to preferred embodiments, the first ADC for use in the inventive method comprises an ADC according to formula (XX) and a second ADC for use in the inventive method according to formula (XII).

According to preferred embodiments, the first ADC for use in the inventive method comprises an ADC according to formula (XX) and a second ADC for use in the inventive method according to formula (XIV)., (XV), or (XX).

According to preferred embodiments, the first ADC for use in the inventive method comprises an ADC according to formula (XX) and a second ADC for use in the inventive method according to formula (XV), or (XX).

According to preferred embodiments, the first ADC for use in the inventive method comprises an ADC according to formula (XX) and a second ADC for use in the inventive method according to formula (XX).

According to some embodiments, the ADC of said first dose of the inventive method as disclosed herein does not specifically bind to a cell surface antigen, tumor-associated antigen or tumor-specific antigen of said patient. The term “cell surface antigen” as used herein refers to a glycan, peptide or protein on the surface of a cell.

The term “tumor-associated antigen” (TAA) as used in the present invention refers to a protein or polypeptide antigen that is expressed by a tumor cell. For example, a TAA may be one or more surface proteins or polypeptides, nuclear proteins or glycoproteins, or fragments thereof, expressed by a tumor cell. For example, human tumor-associated antigens include differentiation antigens (such as melanocyte differentiation antigens), mutational antigens (such as p53), overexpressed cellular antigens (such as HER2), viral antigens (such as human papillomavirus proteins), and cancer/testis (CT) antigens that are expressed in germ cells of the testis and ovary but are silent in normal somatic cells (such as MAGE and NY-ESO-1). Many TAAs are not cancer- or tumor-specific and may also be found on normal tissues and include for example CEA, MAGE, MUC1, surviving, WT1, RNF43, TOMM34, VEGFR-1, VEGFR-2, KOC1, ART4, KRas, EpCAM, HER-2, COA-1 SAP, TGF-βRII, p53, ASCL2, IL13Ralpha2, ASCL2, NY-ESO-1, MAGE-A3, PRAME and SART 1-3, CD37, CD20, CD19, BCMA (CD269), or PSMA.

The term “tumor-specific antigens” (TSAs) as used in the present invention refers to a repertoire of peptides that is displayed on the tumor cell surface and can be specifically recognized by neoantigen-specific T cell receptors (TCRs) in the context of major histocompatibility complexes (MHCs) molecules. TSAs may also be referred to as which are also referred to as “tumor neoantigens” in the context of this invention. From an immunological perspective, tumor neoantigen is the truly foreign protein and entirely absent from normal human organs/tissues. For most human tumors without a viral etiology, tumor neoantigens can e.g. derive from a variety of nonsynonymous genetic alterations including single-nucleotide variants (SNVs), insertions and deletions (indel), gene fusions, frameshift mutations, and structural variants (SVs). The term “tumor-specific antigens” (TSAs) as used according to the invention also includes oncoviral antigens, such as e.g. antigens of human papilloma virus, or Merkel cell polyomavirus (MCPyV). Typically, oncoviral antigens are only found expressed on cells infected with the the respective virus. Tumor-neoantigens may be identified using in silico prediction tools known in the art as disclosed in Trends in Molecular Medicine, November 2019, Pages 980-992.

According to some embodiments, the second ADCs of the inventive method as disclosed herein which is administered to said patient afflicted with cancer as disclosed herein specifically binds to cell surface antigen, tumor-associated antigen or tumor-specific antigen as disclosed herein of said patient.

According to one embodiment, the first and/or second antibody-drug-conjugate for use according to the invention (e.g. for use in the inventive method as disclosed herein) comprises a chimeric antibody.The term “chimeric antibody” refers to those antibodies wherein one portion of each of the amino acid sequences of heavy and light chains is homologous to corresponding sequences in antibodies derived from a particular species or belonging to a particular class, while the remaining segment of the chain is homologous to corresponding sequences in another species or class. Typically the variable region of both light and heavy chains mimics the variable regions of antibodies derived from one species of mammals, while the constant portions are homologous to sequences of antibodies derived from another. One clear advantage to such chimeric forms is that the variable region can conveniently be derived from presently known sources using readily available B-cells or hybridomas from non-human host organisms in combination with constant regions derived from, for example, human cell preparations. While the variable region has the advantage of ease of preparation and the specificity is not affected by the source, the constant region being human is less likely to elicit an immune response from a human subject when the antibodies are injected than would the constant region from a non-human source.

According to a preferred embodiment, the first and/or second antibody-drug-conjugate for use in the inventive method comprises a humanized antibody. A “humanized” antibody refers to an antibody that contains minimal sequences derived from non-human immunoglobulin. Thus, “humanized” forms of non-human (e.g., murine) antibodies are chimeric antibodies that contain minimal sequence derived from the non-human antibody. All or substantially all of the framework (FW) regions may also be those of a human immunoglobulin sequence. The humanized antibody can also contain at least a portion of an immunoglobulin constant region (Fc), typically that of a human immunoglobulin consensus sequence. Methods of antibody humanization are known in the art and have been described, for example, in Riechmann et al., Nature 332:323-7, 1988; U.S. Pat. Nos: 5,530,101; 5,585,089; 5,693,761; 5,693,762; and 6,180,370.

According to a preferred embodiment, the first and/or second antibody-drug-conjugate of the invention comprises a human antibody. The term “human antibody” is intended to include antibodies having variable and constant regions derived from human germline immunoglobulin sequences. A human antibody may include amino acid residues not encoded by human germline immunoglobulin sequences (e.g., mutations introduced by random or site-specific mutagenesis in vitro or during gene rearrangement or by somatic mutation in vivo). However, the term “human antibody”, as used herein, is not intended to include antibodies in which CDR sequences derived from the germline of another mammalian species, such as a mouse, have been grafted onto human framework sequences. A human antibody can be produced in a human cell (for example, by recombinant expression) or by a non-human animal or a prokaryotic or eukaryotic cell that is capable of expressing functionally rearranged human immunoglobulin (such as heavy chain and/or light chain) genes. When a human antibody is a single chain antibody, it can include a linker peptide that is not found in native human antibodies. For example, an Fv can contain a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. Human antibodies can also be produced using transgenic mice that are incapable of expressing functional endogenous immunoglobulins, but which can express human immunoglobulin genes (see, for example, PCT Publication Nos. WO1998/24893; WO1992/01047; WO1996/34096; WO1996/33735; U.S. Pat. Nos. 5,413,923; 5,625,126; 5,633,425; 5,569,825; 5,661,016; 5,545,806; 5,814,318; 5,885,793; 5,916,771; and 5,939,598). In some embodiments, the human antibody of the first and/or second antibody-drug-conjugate is selected from an IgG1, IgG2, or IgG4 isotype, preferably the human antibody of the first and/or second antibody-drug-conjugate is selected from an IgG1 isotype. Accordingly, the antibodies of the first and/or second ADC of the inventive method are preferably monoclonal antibodies. Monoclonal antibodies may e.g. be obtained by recombinant expression in eukaryotic, preferably a yeast, or plant cell, more preferably in a human cell. Methods for manufacturing recombinant antibodies may be done as described in Frenzel et al. Front Immunol. 2013; 4: 217, or e.g. Nat Protoc. 2018 Jan;13(1):99-117.

According to some embodiments, the antibody of the first and/or second ADC for use in the inventive method comprises at least one amino acid substitution in its Fc region. For example, the antibody of the first and/or second ADC of the inventive method comprises at least one of the following amino acid subsitutions in its Fc region from the group of L234A, L234G, L234T, L234Q, L234H; L235A, L235D, L235G, L235H, L235V; L236I, L236N, L236P,L236R, L236G, G237R, G237S, G237T, G237D, G237I, P238A, P238G, P238S, P238T, D265C, P329A,P329G,P329I, P329L,P329M, P329T (according to the EU numbering system, the EU numbering system may also be referred to as “EU index as in Kabat” and refers to the numbering of the human IgG1 EU antibody, which refers to the numbering of the EU antibody of Edelman et al., 1969, Proc Natl Acad Sci USA 63:78-85). Corresponding amino acid subsitutions may be done by genetic engineering which relates to the modification of the amino acid sequence or part thereof of a given or natural polypeptide or protein in the sense of nucleotide and/or amino acid substitution, insertion, deletion or reversion, or any combinations thereof, by gene technological methods such as e.g. site-directed mutagenesis as described in Biochem. J. (1986) 237, 1-7, or J Biol Chem. 2015 Jan 30; 290(5): 2577-2592. As used herein, the term “amino acid substitution” relates to modifications of the amino acid sequence of the protein, wherein one or more amino acids are replaced with the same number of different amino acids, producing a protein which contains a different amino acid sequence than the original protein. A conservative amino acid substitution is understood to relate to a substitution which due to similar size, charge, polarity and/or conformation does not significantly affect the structure and function of the protein. Groups of conservative amino acids in that sense represent, e.g., the non-polar amino acids Gly, Ala, Val, Ile and Leu; the aromatic amino acids Phe, Trp and Tyr; the positively charged amino acids Lys, Arg and His; and the negatively charged amino acids Asp and Glu.

According to preferred embodiments, the antibody of the first and/or second ADC for use in the inventive method comprises at least two amino acid subsitutions in its Fc region, selected from the group comprising

L234A/L235A, L234A/L235D, L234A/L235G, L234A/L235H, L234A/L235V, L234G/L235A, L234G/L235D, L234G/L235G, L234G/L235H, L234G/L235V,L234T/L235A, L234T/L235D, L234T/L235G, L234T/L235H, L234T/L235V, L234A/G237R, L234A/G237S, L234A/G237T, L234A/G237D, L234A/G237I, L234G/G237R, L234A/G237S, L234A/G237T, L234A/G237D, L234A/G237I, L234T/G237R, L234T/G237S, L234T/G237T, L234T/G237D, L234T/G237I, L234A/P238A, L234A/P238G, L234G/P238A, L234G/P238G, L234T/P238A, L234T/P238G, L234A/D265C, L234A/D265C, L234A/D265C, L234G/D265C, L234G/D265C, L234G/D265C, L234T/D265C, L234T/D265C, L234T/D265C, L235A/D265C, L235G/D265C, L235V/D265C (according to EU numbering), more preferably, the least two amino acid subsitutions in the Fc region of the antibody of the first and/or second ADC of the inventive method are selected from the group comprising

L234A/L235A/D265C, L234A/L235D/D265C, L234A/L235G/D265C, L234A/L235H/D265C, L234A/L235V/D265CL234G/L235A/D265C, L234G/L235D/D265C L234G/L235G/D265C, L234G/L235H/D265C, L234G/L235V/D265C, L234T/L235A/D265C, L234T/L235D/D265C, L234T/L235G/D265C, L234T/L235H/D265C, L234T/L235V/D265C, more preferably from the group comprising:

L234A/L235A/D265C, L234A/L235D/D265C, L234A/L235G/D265C, L234G/L235A/D265C, L234G/L235D/D265C L234G/L235G/D265C L234T/L235A/D265C, L234T/L235D/D265C, L234T/L235G/D265C.

According to more preferred embodiments, the Fc region of the antibody of the first and/or second ADC for use in the inventive method comprise at least three amino acid subsitutions selected from the group L234A/L235A/D265C, L234G/L235A/D265C, or L234T/L235A/D265C (according to the EU numbering system), most preferably, the Fc region of the antibody of the first and/or second ADC of the inventive method as disclosed herein comprises the amino acid subsitutuions L234A/L235A/D265C.

The use of ADCs in the inventive method which comprise cysteine-engineered Fc region and comprise e.g. the amino acid substitution D265C (numbering according to the EU system) is particularly useful for coupling amatoxin-linker conjugates which comprise a thiol-reactive moity Z′ for coupling the linker-amatoxin conjugate to the antibody, whereby Z′ may be selected from bromo acetamide, iodo acetamide, methylsulfonylbenzothiazole, 4,6-dichloro-1,3,5-triazin-2-ylamino group methyl-sulfonyl phenyltetrazole or methylsulfonyl phenyloxadiazole, pyridine-2-thiol, 5- nitropyridine-2-thiol, methanethiosulfonate, or preferably a maleimide. The coupling of the construct comprising Z′-L-amatoxin may e.g. be done as disclosed in WO WO2016/142049 A1. The use of antibodies which comprise cysteine-engineered Fc region (e.g. comprising the amino acid substitution D265C) for the manufacture of the first and/or second ADC of the inventive method is advantageous to achieve site-specific antibody-drug conjugates at high yield and homogeneity. In addition, the amino acid subsitutions such as L234A/L235A, L234G/L235A, L234T/L235A decrease the binding of the first and/or second ADC of the inventive method to FcγRI, II, II thereby reducing the effector functions said ADCs which are antibody-dependent cellular cytotoxicity (ADCC) and antibody-dependent cellular phagocytosis (ADCP), as well as Complement-Dependent Cytotoxicity (CDC)). Both, higher purity of the resulting ADCs, e.g. conjugates with a controlled drug-to-antibody-ratio (DAR) of about DAR=2, and the reduction in effector function further improve the therapeutic index (TI) of that can be obtained by the inventive method.

Including additional amino acid subsitutions in addition to D265C such as, e.g. L234A/L235A, L234G/L235A, L234T/L235A into the Fc region of the antibody of the first and/or second ADC may be particularly advantageous since these mutations decreases the binding of the first and/or second ADC of the inventive method to FcγRI, II, II thereby reducing the effector functions of the first and/or second ADC used. The term “effector functions” as used herein hereby refers to antibody-dependent cellular cytotoxicity (ADCC) and antibody-dependent cellular phagocytosis (ADCP), as well as Complement-Dependent Cytotoxicity (CDC)).

It is to be understood that the inventive method of improving the therapeutic index of an ADC is advantageous to improve the TI of the second ADC as used in the inventive method, in case the first ADC is different (e.g. does not bind a tumor antigen, or cell surface antigen, or tumor-specific antigen) from said second ADC. It is further to be understood that the inventive method is particularly useful to increase the TI of amatoxin-based ADCs.

According to one embodiment, the first ADC according to the inventive method as disclosed above is administered administered between about 30, 28, 26, 24, 23 days to about 21, 20, 19, 18, 17, 16, 15 days, or from about 21, 20, 19, 18, to about 17, 16, 15 days prior to the administration of a second dose of a second antibody-drug conjugate, preferably between about 23 days, 21 days to about 20 days, 19 days, 18 days, or 23 days, 22 days, 21 days, 20 days, 19 days, 18 days, prior to the administration of a second dose of a second antibody-drug conjugate.

In some embodiments, the second ADC may be administered more than once, e.g. according to a treatment plan. For example, the second ADC may be administered once a week, or once every 10, 12, 14, 16, 18, 20, 21, 28 days, or once every months, bi-monthly and the like, subsequent to the treatment according to the inventive method.

According to one embodiment, the first dose of the first antibody-drug conjugate of the inventive method may be administered at about 60%, 65%, 70%, 75% to about 80%, 85%, 90%, 100% of the therapeutically effective dose of said second antibody-drug conjugate, preferably the the first antibody-drug-conjugate is dosed at about 65%, 70% to about 75%, 80%, 85%, 90%, 95%, 100%, or of about 85%, 90%, to about 95%, 100% of the the therapeutically effective dose of said second antibody-drug conjugate. The term “therapeutically effective dose” or “therapeutic dose” as used herein is an amount sufficient to effect desired clinical results (i.e., achieve therapeutic efficacy). A therapeutically effective dose can be administered in one or more administrations. For purposes of this invention, a therapeutically effective dose of the first and/or second ADC of the inventive method, preferably of the second ADC of the inventive method is an amount that is sufficient to palliate, ameliorate, stabilize, reverse, prevent, slow or delay the progression of the disease state (e.g., cancer).

According to one aspect, the present invention pertains to antibody-drug-conjugates of the overall structure Ab-(Z-L-Ama)ₙ as disclosed herein for use in the inventive method as disclosed herein. Accordingly, the present invention pertains to antibody-drug-conjugates according to any one of formulae (XII) - (XXII) for use in the inventive method, wherein n is from about 1, 2, 3, 4 to about 5, 6, 7, 8, or from about 5, 6, 7, to about 9, 10, or n is 1, 2, 3, 4, 5, 6, 7, 8, preferably n is between 1 to 4, e.g. 1, 2, 3, 4, more preferably n is from about 1.5, 2.5 to about 3, 3.5 more preferably n is about 1.5 to about 2.5, or preferably n is about 2 for use in the inventive method as disclosed herein.

In one aspect the present invention pertains to a first and second ADC for use in cancer treatment, wherein the first ADC comprises an amatoxin-linker according to any one of formulae (I) to (XI), and wherein the second ADC comprises an amatoxin-linker according to any one of formulae (I) to (XI) as disclosed herein above, and wherein the first ADC for use according to the inventive methode is administered between about 30, 28, 26, 24, 23 days to about 21, 20, 19, 18, 17, 16, 15 days, or from about 21, 20, 19, 18, to about 17, 16, 15 days prior to the administration of a second dose of a second antibody-drug conjugate for use according to the inventive method, preferably between about 23 days, 21 days to about 20 days, 19 days, 18 days, or 23 days, 22 days, 21 days, 20 days, 19 days, 18 days, prior to the administration of a second dose of a second antibody-drug conjugate, whereby the first dose of said first ADC for use according to the inventive method is about 60%, 65%, 70%, 75% to about 80%, 85%, 90%, 100% of the therapeutically effective dose of said second ADC, preferably the dose of said first ADC is about 65%, 70% to about 75%, 80%, 85%, 90%, 95%, 100%, or of about 85%, 90%, to about 95%, 100% of said second dose of said second ADC.

For example, present invention pertains to a first and second ADC for use in cancer treatment, wherein the first ADC comprises an amatoxin-linker according to formula (I), and wherein the second ADC comprises an amatoxin-linker according to any one of formulae (I), (II), (III), (IV), (V), (VI), (VII), (VIII), (IX), (X), or (XI) as disclosed herein above, and wherein the first ADC for use according to the inventive methode is administered between about 30, 28, 26, 24, 23 days to about 21, 20, 19, 18, 17, 16, 15 days, or from about 21, 20, 19, 18, to about 17, 16, 15 days prior to the administration of a second dose of a second antibody-drug conjugate for use according to the inventive method, preferably between about 23 days, 21 days to about 20 days, 19 days, 18 days, or 23 days, 22 days, 21 days, 20 days, 19 days, 18 days, prior to the administration of a second dose of a second antibody-drug conjugate, whereby the first dose of said first ADC for use according to the inventive method is about 60%, 65%, 70%, 75% to about 80%, 85%, 90%, 100% of the therapeutically effective dose of said second ADC, preferably the dose of said first ADC is about 65%, 70% to about 75%, 80%, 85%, 90%, 95%, 100%, or of about 85%, 90%, to about 95%, 100% of said second dose of said second ADC. In one embodiment, the first and second ADC for use in the treatment of cancer are provided as two separate doses, wherein the first dose of said first ADC is a non therapeutic dose and wherein the dose of said second ADC is a therapeutic dose, preferably the first dose of said first ADC for use according to the invention is about 50%, 55%, 60%, 65%, 70%, 75%, 80%, 85%, or from about 50%, 55%, 60% to about 65%, 75%, 80%, or from about 65%, 70%, 75% to about 80%, 85% of the amount of said second dose, whereby said first and second dose of said first and second ADC for use according to the invention are administered at least 10 days, 12 days, 14 days, 16 days, 18 days, 21 days, 28 days, or 35 days apart from each other.

For example, present invention pertains to a first and second ADC for use in cancer treatment, wherein the first ADC comprises an amatoxin-linker according to formula (II), and wherein the second ADC comprises an amatoxin-linker according to any one of formulae (I), (II), (III), (IV), (V), (VI), (VII), (VIII), (IX), (X), or (XI) as disclosed herein above, and wherein the first ADC for use according to the inventive methode is administered between about 30, 28, 26, 24, 23 days to about 21, 20, 19, 18, 17, 16, 15 days, or from about 21, 20, 19, 18, to about 17, 16, 15 days prior to the administration of a second dose of a second antibody-drug conjugate for use according to the inventive method, preferably between about 23 days, 21 days to about 20 days, 19 days, 18 days, or 23 days, 22 days, 21 days, 20 days, 19 days, 18 days, prior to the administration of a second dose of a second antibody-drug conjugate, whereby the first dose of said first ADC for use according to the inventive method is about 60%, 65%, 70%, 75% to about 80%, 85%, 90%, 100% of the therapeutically effective dose of said second ADC, preferably the dose of said first ADC is about 65%, 70% to about 75%, 80%, 85%, 90%, 95%, 100%, or of about 85%, 90%, to about 95%, 100% of said second dose of said second ADC. In one embodiment, the first and second ADC for use in the treatment of cancer are provided as two separate doses, wherein the first dose of said first ADC is a non therapeutic dose and wherein the dose of said second ADC is a therapeutic dose, preferably the first dose of said first ADC for use according to the invention is about 50%, 55%, 60%, 65%, 70%, 75%, 80%, 85%, or from about 50%, 55%, 60% to about 65%, 75%, 80%, or from about 65%, 70%, 75% to about 80%, 85% of the amount of said second dose, whereby said first and second dose of said first and second ADC for use according to the invention are administered at least 10 days, 12 days, 14 days, 16 days, 18 days, 21 days, 28 days, or 35 days apart from each other.

For example, present invention pertains to a first and second ADC for use in cancer treatment, wherein the first ADC comprises an amatoxin-linker according to formula (III), and wherein the second ADC comprises an amatoxin-linker according to any one of formulae (I), (II), (III), (IV), (V), (VI), (VII), (VIII), (IX), (X), or (XI) as disclosed herein above, and wherein the first ADC for use according to the inventive methode is administered between about 30, 28, 26, 24, 23 days to about 21, 20, 19, 18, 17, 16, 15 days, or from about 21, 20, 19, 18, to about 17, 16, 15 days prior to the administration of a second dose of a second antibody-drug conjugate for use according to the inventive method, preferably between about 23 days, 21 days to about 20 days, 19 days, 18 days, or 23 days, 22 days, 21 days, 20 days, 19 days, 18 days, prior to the administration of a second dose of a second antibody-drug conjugate, whereby the first dose of said first ADC for use according to the inventive method is about 60%, 65%, 70%, 75% to about 80%, 85%, 90%, 100% of the therapeutically effective dose of said second ADC, preferably the dose of said first ADC is about 65%, 70% to about 75%, 80%, 85%, 90%, 95%, 100%, or of about 85%, 90%, to about 95%, 100% of said second dose of said second ADC. In one embodiment, the first and second ADC for use in the treatment of cancer are provided as two separate doses, wherein the first dose of said first ADC is a non therapeutic dose and wherein the dose of said second ADC is a therapeutic dose, preferably the first dose of said first ADC for use according to the invention is about 50%, 55%, 60%, 65%, 70%, 75%, 80%, 85%, or from about 50%, 55%, 60% to about 65%, 75%, 80%, or from about 65%, 70%, 75% to about 80%, 85% of the amount of said second dose, whereby said first and second dose of said first and second ADC for use according to the invention are administered at least 10 days, 12 days, 14 days, 16 days, 18 days, 21 days, 28 days, or 35 days apart from each other.

In one aspect the present invention pertains to a first and second ADC for use in cancer treatment, wherein the first ADC is according to any one of formulae (XII) to (XXII), e.g. (XII), (XIII), (XIV), (XV), (XVI), (XVII), (XVlll), (XIX), (XX), (XXI), (XXII) and wherein the second ADC is according to any of formulae (XII) to (XXII), e.g. (XII), (XIII), (XIV), (XV), (XVI), (XVII), (XVIII), (XIX), (XX), (XXI), (XXII) as disclosed herein above, and wherein the first ADC for use according to the inventive methode is administered between about 30, 28, 26, 24, 23 days to about 21, 20, 19, 18, 17, 16, 15 days, or from about 21, 20, 19, 18, to about 17, 16, 15 days prior to the administration of a second dose of a second antibody-drug conjugate for use according to the inventive method, preferably between about 23 days, 21 days to about 20 days, 19 days, 18 days, or 23 days, 22 days, 21 days, 20 days, 19 days, 18 days, prior to the administration of a second dose of a second antibody-drug conjugate, whereby the first dose of said first ADC for use according to the inventive method is about 60%, 65%, 70%, 75% to about 80%, 85%, 90%, 100% of the therapeutically effective dose of said second ADC, preferably the dose of said first ADC is about 65%, 70% to about 75%, 80%, 85%, 90%, 95%, 100%, or of about 85%, 90%, to about 95%, 100% of said second dose of said second ADC, wherein the antibody binds to any of the tumor-associated antigens (TAA) as disclosed herein above.

For example, the present invention pertains to a first and second ADC for use in cancer treatment, wherein the first ADC is according to formula (XII) and wherein the second ADC is according to any of formulae (XII) to (XXII), e.g. (XII), (XIII), (XIV), (XV), (XVI), (XVII), (XVIII), (XIX), (XX), (XXI), (XXII) as disclosed herein above, and wherein the first ADC for use according to the inventive methode is administered between about 30, 28, 26, 24, 23 days to about 21, 20, 19, 18, 17, 16, 15 days, or from about 21, 20, 19, 18, to about 17, 16, 15 days prior to the administration of a second dose of a second antibody-drug conjugate for use according to the inventive method, preferably between about 23 days, 21 days to about 20 days, 19 days, 18 days, or 23 days, 22 days, 21 days, 20 days, 19 days, 18 days, prior to the administration of a second dose of a second antibody-drug conjugate, whereby the first dose of said first ADC for use according to the inventive method is about 60%, 65%, 70%, 75% to about 80%, 85%, 90%, 100% of the therapeutically effective dose of said second ADC, preferably the dose of said first ADC is about 65%, 70% to about 75%, 80%, 85%, 90%, 95%, 100%, or of about 85%, 90%, to about 95%, 100% of said second dose of said second ADC, wherein the antibody binds to any of the tumor-associated antigens (TAA) as disclosed herein above.

For example, the present invention pertains to a first and second ADC for use in cancer treatment, wherein the first ADC is according to formula (XIV) and wherein the second ADC is according to any of formulae (XII) to (XXII), e.g. (XII), (XIII), (XIV), (XV), (XVI), (XVII), (XVIII), (XIX), (XX), (XXI), (XXII) as disclosed herein above, and wherein the first ADC for use according to the inventive methode is administered between about 30, 28, 26, 24, 23 days to about 21, 20, 19, 18, 17, 16, 15 days, or from about 21, 20, 19, 18, to about 17, 16, 15 days prior to the administration of a second dose of a second antibody-drug conjugate for use according to the inventive method, preferably between about 23 days, 21 days to about 20 days, 19 days, 18 days, or 23 days, 22 days, 21 days, 20 days, 19 days, 18 days, prior to the administration of a second dose of a second antibody-drug conjugate, whereby the first dose of said first ADC for use according to the inventive method is about 60%, 65%, 70%, 75% to about 80%, 85%, 90%, 100% of the therapeutically effective dose of said second ADC, preferably the dose of said first ADC is about 65%, 70% to about 75%, 80%, 85%, 90%, 95%, 100%, or of about 85%, 90%, to about 95%, 100% of said second dose of said second ADC, wherein the antibody binds to any of the tumor-associated antigens (TAA) as disclosed herein above.

For example, the present invention pertains to a first and second ADC for use in cancer treatment, wherein the first ADC is according to formula (XX) and wherein the second ADC is according to any of formulae (XII) to (XXII), e.g. (XII), (XIII), (XIV), (XV), (XVI), (XVII), (XVIII), (XIX), (XX), (XXI), (XXII) as disclosed herein above, and wherein the first ADC for use according to the inventive methode is administered between about 30, 28, 26, 24, 23 days to about 21, 20, 19, 18, 17, 16, 15 days, or from about 21, 20, 19, 18, to about 17, 16, 15 days prior to the administration of a second dose of a second antibody-drug conjugate for use according to the inventive method, preferably between about 23 days, 21 days to about 20 days, 19 days, 18 days, or 23 days, 22 days, 21 days, 20 days, 19 days, 18 days, prior to the administration of a second dose of a second antibody-drug conjugate, whereby the first dose of said first ADC for use according to the inventive method is about 60%, 65%, 70%, 75% to about 80%, 85%, 90%, 100% of the therapeutically effective dose of said second ADC, preferably the dose of said first ADC is about 65%, 70% to about 75%, 80%, 85%, 90%, 95%, 100%, or of about 85%, 90%, to about 95%, 100% of said second dose of said second ADC, wherein the antibody binds to any of the tumor-associated antigens (TAA) as disclosed herein above.

For example, the present invention pertains to a first and second ADC for use in cancer treatment, wherein the first ADC is according to formula (XX) and wherein the second ADC is according to formula (XII) as disclosed herein above, and wherein the first ADC for use according to the inventive methode is administered between about 30, 28, 26, 24, 23 days to about 21, 20, 19, 18, 17, 16, 15 days, or from about 21, 20, 19, 18, to about 17, 16, 15 days prior to the administration of a second dose of a second antibody-drug conjugate for use according to the inventive method, preferably between about 23 days, 21 days to about 20 days, 19 days, 18 days, or 23 days, 22 days, 21 days, 20 days, 19 days, 18 days, prior to the administration of a second dose of a second antibody-drug conjugate, whereby the first dose of said first ADC for use according to the inventive method is about 60%, 65%, 70%, 75% to about 80%, 85%, 90%, 100% of the therapeutically effective dose of said second ADC, preferably the dose of said first ADC is about 65%, 70% to about 75%, 80%, 85%, 90%, 95%, 100%, or of about 85%, 90%, to about 95%, 100% of said second dose of said second ADC, wherein the antibody binds to any of the tumor-associated antigens (TAA) as disclosed herein above.

For example, the present invention pertains to a first and second ADC for use in cancer treatment, wherein the first ADC is according to formula (XX) and wherein the second ADC is according to formula (XIV) as disclosed herein above, and wherein the first ADC for use according to the inventive methode is administered between about 30, 28, 26, 24, 23 days to about 21, 20, 19, 18, 17, 16, 15 days, or from about 21, 20, 19, 18, to about 17, 16, 15 days prior to the administration of a second dose of a second antibody-drug conjugate for use according to the inventive method, preferably between about 23 days, 21 days to about 20 days, 19 days, 18 days, or 23 days, 22 days, 21 days, 20 days, 19 days, 18 days, prior to the administration of a second dose of a second antibody-drug conjugate, whereby the first dose of said first ADC for use according to the inventive method is about 60%, 65%, 70%, 75% to about 80%, 85%, 90%, 100% of the therapeutically effective dose of said second ADC, preferably the dose of said first ADC is about 65%, 70% to about 75%, 80%, 85%, 90%, 95%, 100%, or of about 85%, 90%, to about 95%, 100% of said second dose of said second ADC, wherein the antibody binds to any of the tumor-associated antigens (TAA) as disclosed herein above.

For example, the present invention pertains to a first and second ADC for use in cancer treatment, wherein the first ADC is according to formula (XX) and wherein the second ADC is according to formula (XX) as disclosed herein above, and wherein the first ADC for use according to the inventive methode is administered between about 30, 28, 26, 24, 23 days to about 21, 20, 19, 18, 17, 16, 15 days, or from about 21, 20, 19, 18, to about 17, 16, 15 days prior to the administration of a second dose of a second antibody-drug conjugate for use according to the inventive method, preferably between about 23 days, 21 days to about 20 days, 19 days, 18 days, or 23 days, 22 days, 21 days, 20 days, 19 days, 18 days, prior to the administration of a second dose of a second antibody-drug conjugate, whereby the first dose of said first ADC for use according to the inventive method is about 60%, 65%, 70%, 75% to about 80%, 85%, 90%, 100% of the therapeutically effective dose of said second ADC, preferably the dose of said first ADC is about 65%, 70% to about 75%, 80%, 85%, 90%, 95%, 100%, or of about 85%, 90%, to about 95%, 100% of said second dose of said second ADC, wherein the antibody binds to any of the tumor-associated antigens (TAA) as disclosed herein above.

For example, the present invention pertains to a first and second ADC for use in cancer treatment, wherein the first ADC is according to formula (XII) and wherein the second ADC is according to formula (XII) as disclosed herein above, and wherein the first ADC for use according to the inventive methode is administered between about 30, 28, 26, 24, 23 days to about 21, 20, 19, 18, 17, 16, 15 days, or from about 21, 20, 19, 18, to about 17, 16, 15 days prior to the administration of a second dose of a second antibody-drug conjugate for use according to the inventive method, preferably between about 23 days, 21 days to about 20 days, 19 days, 18 days, or 23 days, 22 days, 21 days, 20 days, 19 days, 18 days, prior to the administration of a second dose of a second antibody-drug conjugate, whereby the first dose of said first ADC for use according to the inventive method is about 60%, 65%, 70%, 75% to about 80%, 85%, 90%, 100% of the therapeutically effective dose of said second ADC, preferably the dose of said first ADC is about 65%, 70% to about 75%, 80%, 85%, 90%, 95%, 100%, or of about 85%, 90%, to about 95%, 100% of said second dose of said second ADC, wherein the antibody binds to any of the tumor-associated antigens (TAA) as disclosed herein above.

For example, the present invention pertains to a first and second ADC for use in cancer treatment, wherein the first ADC is according to formula (XII) and wherein the second ADC is according to formula (XX) as disclosed herein above, and wherein the first ADC for use according to the inventive methode is administered between about 30, 28, 26, 24, 23 days to about 21, 20, 19, 18, 17, 16, 15 days, or from about 21, 20, 19, 18, to about 17, 16, 15 days prior to the administration of a second dose of a second antibody-drug conjugate for use according to the inventive method, preferably between about 23 days, 21 days to about 20 days, 19 days, 18 days, or 23 days, 22 days, 21 days, 20 days, 19 days, 18 days, prior to the administration of a second dose of a second antibody-drug conjugate, whereby the first dose of said first ADC for use according to the inventive method is about 60%, 65%, 70%, 75% to about 80%, 85%, 90%, 100% of the therapeutically effective dose of said second ADC, preferably the dose of said first ADC is about 65%, 70% to about 75%, 80%, 85%, 90%, 95%, 100%, or of about 85%, 90%, to about 95%, 100% of said second dose of said second ADC, wherein the antibody binds to any of the tumor-associated antigens (TAA) as disclosed herein above.

For example, the present invention pertains to a first and second ADC for use in cancer treatment, wherein the first ADC is according to formula (XII) and wherein the second ADC is according to formula (XIV) as disclosed herein above, and wherein the first ADC for use according to the inventive methode is administered between about 30, 28, 26, 24, 23 days to about 21, 20, 19, 18, 17, 16, 15 days, or from about 21, 20, 19, 18, to about 17, 16, 15 days prior to the administration of a second dose of a second antibody-drug conjugate for use according to the inventive method, preferably between about 23 days, 21 days to about 20 days, 19 days, 18 days, or 23 days, 22 days, 21 days, 20 days, 19 days, 18 days, prior to the administration of a second dose of a second antibody-drug conjugate, whereby the first dose of said first ADC for use according to the inventive method is about 60%, 65%, 70%, 75% to about 80%, 85%, 90%, 100% of the therapeutically effective dose of said second ADC, preferably the dose of said first ADC is about 65%, 70% to about 75%, 80%, 85%, 90%, 95%, 100%, or of about 85%, 90%, to about 95%, 100% of said second dose of said second ADC, wherein the antibody binds to any of the tumor-associated antigens (TAA) as disclosed herein above.

For example, the present invention pertains to a first and second ADC for use in cancer treatment, wherein the first ADC is according to formula (XIV) and wherein the second ADC is according to formula (XIV) as disclosed herein above, and wherein the first ADC for use according to the inventive methode is administered between about 30, 28, 26, 24, 23 days to about 21, 20, 19, 18, 17, 16, 15 days, or from about 21, 20, 19, 18, to about 17, 16, 15 days prior to the administration of a second dose of a second antibody-drug conjugate for use according to the inventive method, preferably between about 23 days, 21 days to about 20 days, 19 days, 18 days, or 23 days, 22 days, 21 days, 20 days, 19 days, 18 days, prior to the administration of a second dose of a second antibody-drug conjugate, whereby the first dose of said first ADC for use according to the inventive method is about 60%, 65%, 70%, 75% to about 80%, 85%, 90%, 100% of the therapeutically effective dose of said second ADC, preferably the dose of said first ADC is about 65%, 70% to about 75%, 80%, 85%, 90%, 95%, 100%, or of about 85%, 90%, to about 95%, 100% of said second dose of said second ADC, wherein the antibody binds to any of the tumor-associated antigens (TAA) as disclosed herein above.

According to a preferred embodiment, the first and second ADC for use in the treatment of cancer comprise the ADC according to formula (I) (SEQ ID NO:1) as disclosed in WO2018/115466 A1, wherein the antibody “J22.9-ISY-D265C” designates an anti-BCMA antibody comprising a heavy chain amino acid sequence according to SEQ ID NO: 1 and a light chain amino acid sequence according to SEQ ID NO: 2 as disclosed in WO2018/115466:

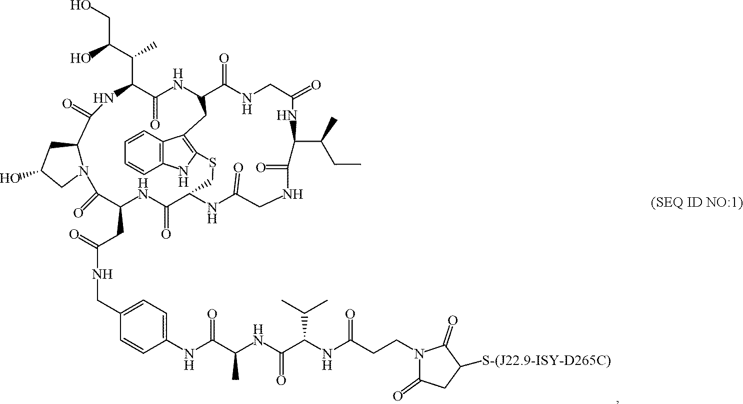

wherein the cancer is selected from the group comprising multiple myeloma, diffuse large B-cell lymphoma (DLBCL), and chronic lymphocytic leukemia (CLL), particularly multiple myeloma. Thus, the first and second ADC for use according to the invention are the same ADC wherein the first dose of said ADC is is about 50%, 55%, 60%, 65%, 70%, 75%, 80%, 85%, or from about 50%, 55%, 60% to about 65%, 75%, 80%, or from about 65%, 70%, 75% to about 80%, 85% of the amount of said second dose, whereby said first and second dose of said first and second ADC for use according to the invention are administered at least 10 days, 12 days, 14 days, 16 days, 18 days, 21 days, 28 days, or 35 days apart from each other.

According to a preferred embodiment, the first and second ADC for use in the treatment of cancer comprise an anti-CD37 ADC as disclosed in WO 2022/194988 A2 which consist of the anti-CD37 antibody chHH1-HDPLALA-D266C which comprises a heavy chain amino acid sequence according to SEQ ID NO: 11 and a light chain amino acid sequence according to SEQ ID NO: 12, whereby said ADC is according to one of formulae (XII), (XIV), (XV), or (XX), and wherein the cancer is selected from non-Hodgkin’s lymphoma (NHL), follicular lymphoma, diffuse large B cell non-Hodgkin’s lymphoma (DBNHL), subtypes of non-Hodgkin’s lymphoma including mantle cell lymphoma (MCL), chronic lymphocytic leukaemia (CLL), Richter syndrome, primary cutaneous marginal zone lymphoma (PCMZL), hairy cell leukemia, acute myeloid leukemia (AML), rheumatoid arthritis, granulomatosis with polyangiitis and microscopic polyangiitis and pemphigus vulgaris.. Thus, the first and second ADC for use in the treatment of cancer according to the invention are the same ADC wherein the first dose of said ADC is is about 50%, 55%, 60%, 65%, 70%, 75%, 80%, 85%, or from about 50%, 55%, 60% to about 65%, 75%, 80%, or from about 65%, 70%, 75% to about 80%, 85% of the amount of said second dose, whereby said first and second dose of said first and second ADC for use according to the invention are administered at least 10 days, 12 days, 14 days, 16 days, 18 days, 21 days, 28 days, or 35 days apart from each other.

According to a preferred embodiment, the first and second ADC for use in the treatment of cancer comprise an anti-PSMA ADC as disclosed in WO 2020/025564, wherein the antibody of said ADC is one of 3-F11-var1, 3-F11-var2, 3-F11-var3, 3-F11-var4, 3-F11-var5, 3-F11-var6, 3-F11-var7, 3-F11-var8, 3-F11-var9, 3-F11-var10, 3-F11-var11, 3-F11-var12, 3-F11-var13, 3-F11-var14, 3-F11-var115, or 3-F11-var16 and wherein said anti-PSMA ADC has the structure according to one of formulae (XII), (XIV), (XV), or (XX), wherein the cancer is selected from the group comprising prostate cancer, castration-resistant prostate cancer. Accordingly, the first and second ADC for use in the treatment of cancer according to the invention are the same ADC wherein the first dose of said ADC is is about 50%, 55%, 60%, 65%, 70%, 75%, 80%, 85%, or from about 50%, 55%, 60% to about 65%, 75%, 80%, or from about 65%, 70%, 75% to about 80%, 85% of the amount of said second dose, whereby said first and second dose of said first and second ADC for use according to the invention are administered at least 10 days, 12 days, 14 days, 16 days, 18 days, 21 days, 28 days, or 35 days apart from each other.

For example, the first and second ADC for use in the treatment of cancer comprise an anti-PSMA ADC as disclosed in WO 2020/025564, wherein the antibody of said ADC is one of 3-F11-var1, 3-F11-var2, 3-F11-var3, 3-F11-var4, 3-F11-var5, 3-F11-var6, 3-F11-var7, 3-F11-var8, 3-F11-var9, 3-F11-var10, 3-F11-var11, 3-F11-var12, 3-F11-var13, 3-F11-var14, 3-F11-var115, or 3-F11-var16 and wherein said anti-PSMA ADC has the structure according to formula (XIV) and wherein the cancer is selected from the group comprising prostate cancer, castration-resistant prostate cancer. Accordingly, the first and second ADC for use in the treatment of cancer according to the invention are the same ADC wherein the first dose of said ADC is is about 50%, 55%, 60%, 65%, 70%, 75%, 80%, 85%, or from about 50%, 55%, 60% to about 65%, 75%, 80%, or from about 65%, 70%, 75% to about 80%, 85% of the amount of said second dose, whereby said first and second dose of said first and second ADC for use according to the invention are administered at least 10 days, 12 days, 14 days, 16 days, 18 days, 21 days, 28 days, or 35 days apart from each other.

For example, the first and second ADC for use in the treatment of cancer comprise an anti-PSMA ADC as disclosed in WO 2020/025564, wherein the antibody of said ADC is one of 3-F11-var1, 3-F11-var2, 3-F11-var3, 3-F11-var4, 3-F11-var5, 3-F11-var6, 3-F11-var7, 3-F11-var8, 3-F11-var9, 3-F11-var10, 3-F11-var11, 3-F11-var12, 3-F11-var13, 3-F11-var14, 3-F11-var115, or 3-F11-var16 and wherein said anti-PSMA ADC has the structure according to formula (XX) and wherein the cancer is selected from the group comprising prostate cancer, castration-resistant prostate cancer. Accordingly, the first and second ADC for use in the treatment of cancer according to the invention are the same ADC wherein the first dose of said ADC is is about 50%, 55%, 60%, 65%, 70%, 75%, 80%, 85%, or from about 50%, 55%, 60% to about 65%, 75%, 80%, or from about 65%, 70%, 75% to about 80%, 85% of the amount of said second dose, whereby said first and second dose of said first and second ADC for use according to the invention are administered at least 10 days, 12 days, 14 days, 16 days, 18 days, 21 days, 28 days, or 35 days apart from each other.

For example, the first and second ADC for use in the treatment of cancer comprise an anti-PSMA ADC as disclosed in WO 2020/025564, wherein the antibody of said ADC is one of 3-F11-var1, 3-F11-var2, 3-F11-var3, 3-F11-var4, 3-F11-var5, 3-F11-var6, 3-F11-var7, 3-F11-var8, 3-F11-var9, 3-F11-var10, 3-F11-var11, 3-F11-var12, 3-F11-var13, 3-F11-var14, 3-F11-var115, or 3-F11-var16 and wherein said anti-PSMA ADC has the structure according to formula (XII) and wherein the cancer is selected from the group comprising prostate cancer, castration-resistant prostate cancer. Accordingly, the first and second ADC for use in the treatment of cancer according to the invention are the same ADC wherein the first dose of said ADC is is about 50%, 55%, 60%, 65%, 70%, 75%, 80%, 85%, or from about 50%, 55%, 60% to about 65%, 75%, 80%, or from about 65%, 70%, 75% to about 80%, 85% of the amount of said second dose, whereby said first and second dose of said first and second ADC for use according to the invention are administered at least 10 days, 12 days, 14 days, 16 days, 18 days, 21 days, 28 days, or 35 days apart from each other.

In some aspects, the inventive method as disclosed herein comprises repeated dosing, of the second amatoxin-based ADC as disclosed herein (e.g. the second amatoxin-based ADC for use in the inventive method as disclosed herein) subsequent to the second dose, wherein any subsequent therapeutic dose is administered at least 10 days, 12 days, 14 days, 16, days, 18 days, 21 days, 28 days, or 35 days after the second or any subsequent dose.

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Example 1: Mouse Studies

### Synthesis of Amatoxin-Based Antibody-Drug-Conjuagtes

Anti-BCMA, anti-PSMA and anti-digoxigenin (DIG) antibodies were produced by Heidelberg Pharma Research GmbH (HDP) as disclosed in WO2018/115466 A1 and WO2020/216947 A1. Cysteine-reactive amanitin-linker constructs, synthesized at HDP, were conjugated site-specifically to cysteine-engineered antibodies as described in WO2016/142049 A1. Drug-antibody ratio (DAR) according to LC-MS analysis was ~2.0 amanitins per IgG.

### Mouse Tolerability Study

Female CB17 SCID mice were treated with 10 or 25 mg/kg of an anti-DIG ATAC or PBS as single intravenous (i.v.) dose on day 0. On day 21, all mice were treated with 40 or 50 mg/kg of the same amatoxin-based ADC.

### Efficacy Study in LNCaP Prostate Xenograft Model

2.5×10⁶ LNCaP cells (Horoszewicz et al., Cancer Res. 1983 Apr;43(4):1809-18) were implanted s.c. into male CB17 SCID mice. Once a mean tumor volume of 97 mm³ was reached, mice were randomized into groups of 15 mice and were treated i.v. with PBS or an amatoxin-based anti-PSMA ADC either as single dose or in a q7dx4 treatment regiment.

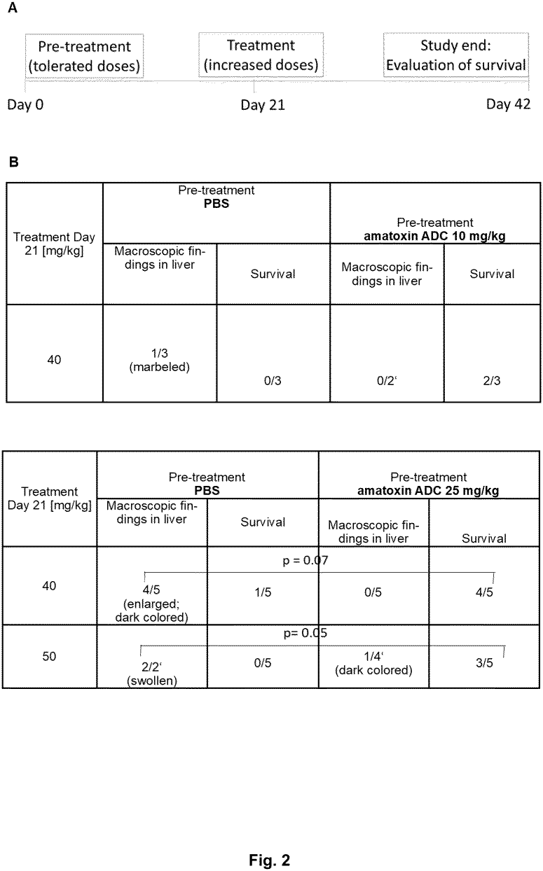

The impact of repeated dosing on the tolerability of amatoxin-based ADCs was investigated in immuno-compromised mice. Previously, an anti-digoxigenen (DIG) amatoxin conjugate was found to be tolerated up to 30 mg/kg as single dose i.v. treatment in naïve mice. Based on this finding, mice were pre-treated with a tolerated dose of 10 or 25 mg/kg of the anti-DIG amatoxin-based ADC followed by a second higher dose of the same ADC of 40 or 50 mg/kg 21 days later. Survival of the mice and macroscopic liver changes were analyzed (FIG. 2).

### Results

Pre-treatment with a tolerated dose of an anti-DIG amatoxin-based ADC improves the tolerability of a higher dose of the same conjugate that is toxic if applied at single dose in naïve animals as seen by less macroscopic liver changes and improved survival in pretreated animals.

### Example 2: Liver Toxicity Studies in Cynomolgus Monkeys

Non-human primates are the most relevant species for toxicity testing of amatoxin-based ADCs as accepted by US and European authorities. Therefore, toxicity was assessed in cynomolgus monkeys.

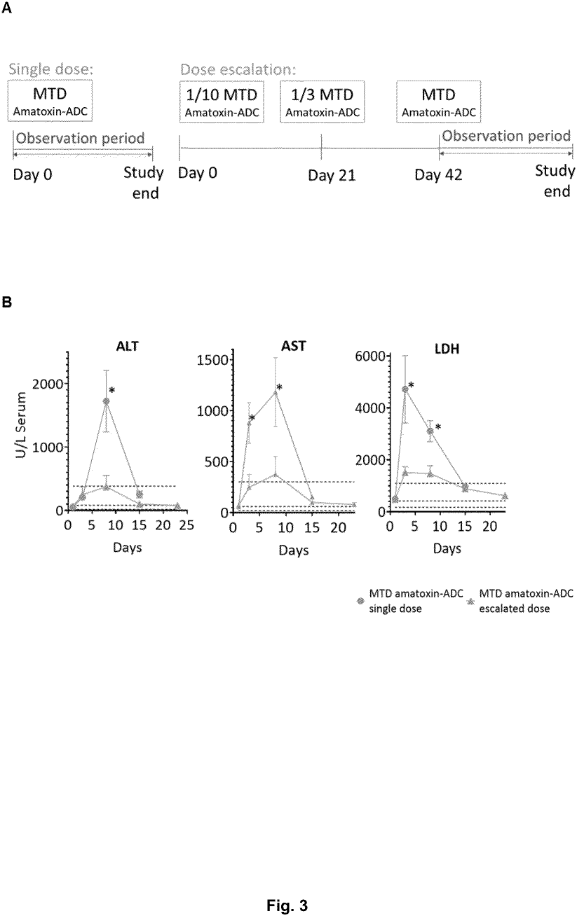

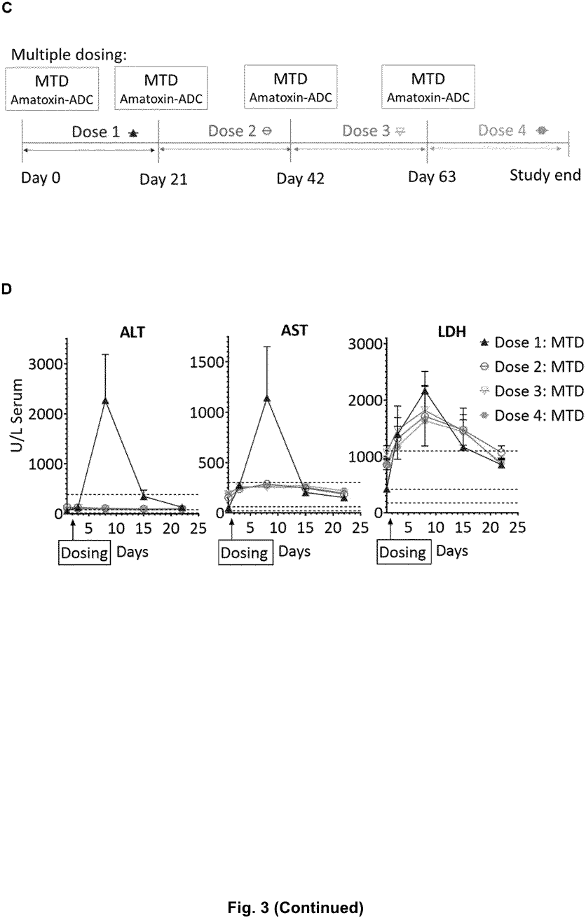

Studies in Cynomolgus monkeys were performed at LPT or AltaSciences. 2-to-4-year-old monkeys were treated with the amatoxin-based anti-BCMA ADC according to Formlua I of WO2018/115466 A1 (referred to as “Amatoxin-ADC” in FIG. 3) via a 30 min i.v. infusion as single dose, in a multiple dose or escalating dose treatment scheme. Blood was collected pre-dose, 3 days, 7 days, 14 days and 21 days after the treatment. LDH, AST and ALT levels in the serum were measured using the methods recommended by the IFCC.

### Results

Treatment with amatoxin-ADC leads to reduced levels of the liver damage markers ALT, AST and LDH as depicted in FIG. 3D in cynomolgus monkeys if applied either following a dose escalation treatment of as second, third or fourth treatment round as compared to single and first treatment, respectively. This data indicate that multiple dose treatment increases the tolerability of amatoxin-based ADCs also in cynomolgus monkeys.

### Example 3: Multiple Dosing Increases the Anti-Tumor Efficacy of Amatoxin- Based ADCs

In view of the results of Examples 1, 2 that repeated treatment was illustrated to improve tolerability of amatoxin-based ADCs in mice and cynomolgus monkeys, it was investigated whether multiple dosing affects the anit-tumor efficacy of amatoxin-based ADCs.

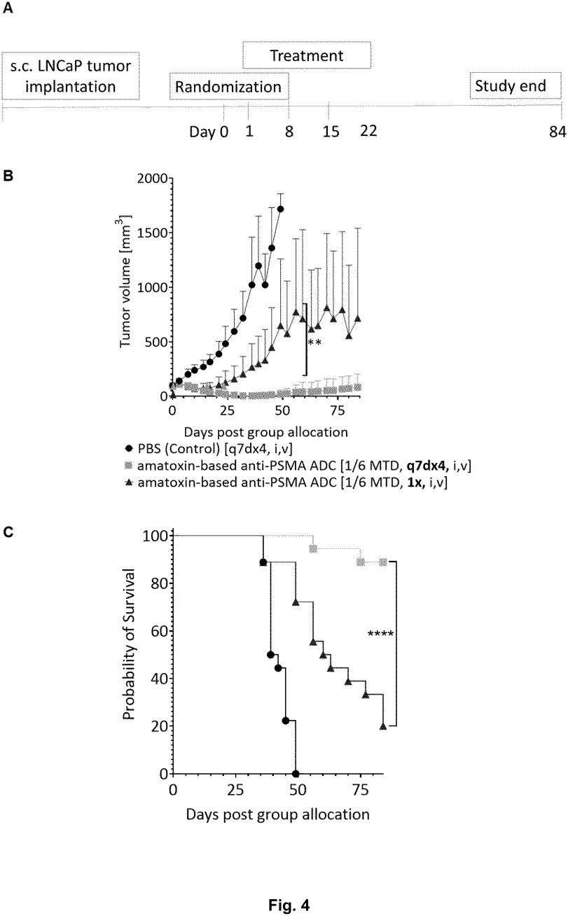

Mice bearing subcutaneous (s.c.) human prostate adenocarcinoma LNCaP tumors were treated according to the treatment regime depicted in FIG. 4A with a single dose or repeated doses (q7dx4) of an amatoxin-based anti-PSMA ADC. Tumor growth and survival was analyzed as shown in FIG. 4B

The results indicate that multiple dosing (q7dx4) in accordance with the inventive method improves the anti-tumor effect of an amatoxin-based anti-PSMA ADC in a s.c. human prostate adenocarcinoma LNCaP CDX model significantly as compared to single dose treatment as demonstrated by reduced tumor growth and improved survival of the mice.

### Example 4: Conjugation of Amatoxin-Linker Payloads

Antibodies were conjugated to the amatoxin linker conjugates by means of the so-called Thiomab technology. In this approach, the conjugation takes place by conjugation of the maleimide residue of the toxin linker construct to the free SH group of a cysteine residue in the antibody, as shown in the following reaction scheme:

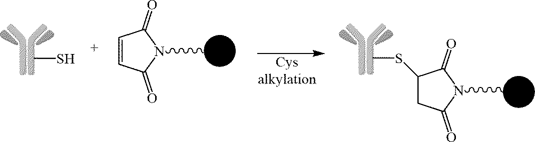

The principles of this conjugation method, are disclosed in Junutula et al (2008) Nat Biotechnology Vol. 26: 925-932.

The antibodies used in the present experiments comprise a D265C substitution in both Fc domains, in order to provide a cystein residue that has such free SH group. The respective technology is disclosed in WO2016/142049 A1, the content of which is incorporated herein by reference, and which results in a homogenous product with a fixed drug to antibody ration (“DAR”) of 2 and a site specific conjugation.

### REFERENCES

Carl et al., J. Med. Chem. (1981) 24:479-480
Chakravarty et al (1983) J. Med. Chem. 26:638-644
“Compendium of Chemical Terminology” published by the International Union of Pure and Applied Chemistry,ISBN: 0-9678550-9-8.
de Groot et al (2001) J. Org. Chem. 66:8815-8830
Dubowchik and Walker, 1999, Pharm. Therapeutics 83:67-123
Edelman, G.M. et al., Proc. Natl. Acad. USA, 63, 78-85 (1969)
Hay et al. (1999) Bioorg. Med. Chem. Lett. 9:2237
Hermanson, G. T. (1996) Bioconjugate Techniques; Academic Press: New York, p. 234-242) Jain et al., Pharm. Res. 32:3526-3540, 2015
Junutula et al (2008) Nat Biotechnology Vol. 26: 925-932.
Leriche et al., Bioorg. Med. Chem., 20:571-582, 2012
Malmqvist M., Curr Opin Immunol. 1993 Apr;5(2):282-6
Neville et al., 1989, Biol. Chem. 264:14653-14661
Thorpe et al., 1987, Cancer Res. 47:5924-5931
Voss et al. BMC Molecular Biology 2014, 15:7
Wawrzynczak et al., In Immunoconjugates: Antibody Conjugates in Radioimagery and Therapy of Cancer (C. W. Vogel ed., Oxford U. Press, 1987
Wieland, T. and Faulstich H. (Wieland T, Faulstich H., CRC Crit Rev Biochem. 5 (1978) 185-260)

1. A method of improving the therapeutic index of an antibody-drug-conjugate, wherein the method comprises the step of:
administering to a patient afflicted with cancer a first dose of a first antibody-drug-conjugate, wherein said first dose of said first antibody-drug conjugate is administered between about 30 days to about 15 days prior to the administration of a second dose of a second antibody-drug conjugate.

2. The method according to claim 1, wherein said first and second dose comprise the same antibody-drug conjugate.

3. The method according to claim 1, wherein said first and second dose comprise a different antibody-drug conjugate.

4. The method according claim 1, wherein said ADC of said first and second dose comprises an RNA polymerase II inhibitor.

5. The method according to claim 4, wherein RNA polymerase II inhibitor is selected from the group of amatoxins.

6. The method according to claim 5, wherein the first and/or second antibody drug conjugate have the structure of 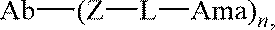
wherein an antibody or antigen-binding fragment thereof (Ab) is conjugated (covalently linked) to linker (L), through a chemical moiety (Z), to an amatoxin moiety (Ama) and n is from about 1 to about 10.

7. The method according to claim 6, wherein the linker is a cleavable or a non-cleavable linker.

8. The method according to claim 6, wherein the amatoxin-linker conjugate Ama-L is selected from the group comprising conjugates (I) to (XI): 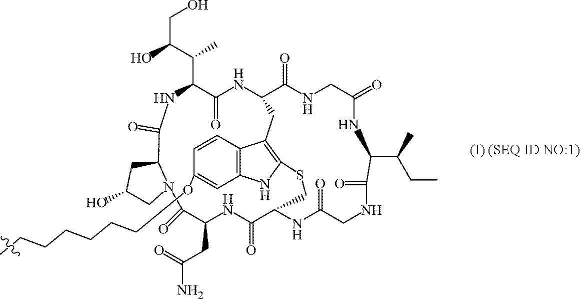 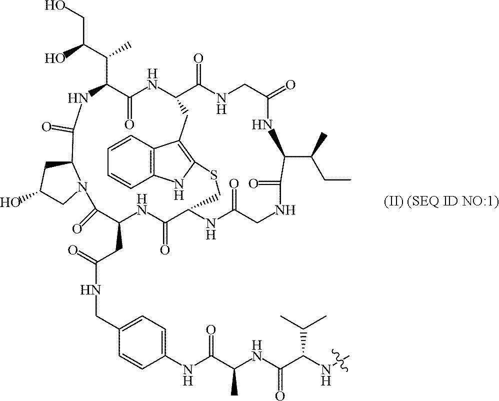 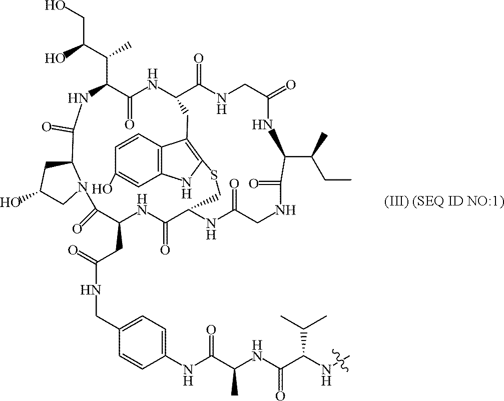 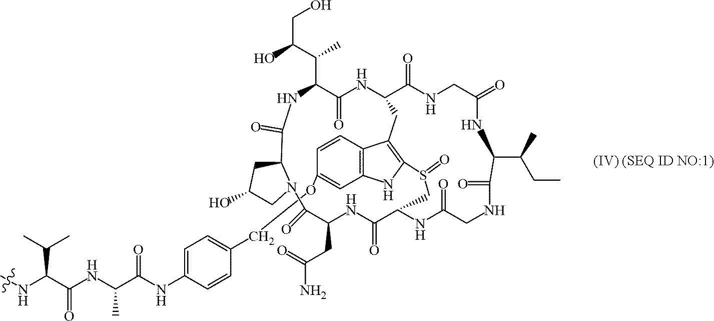 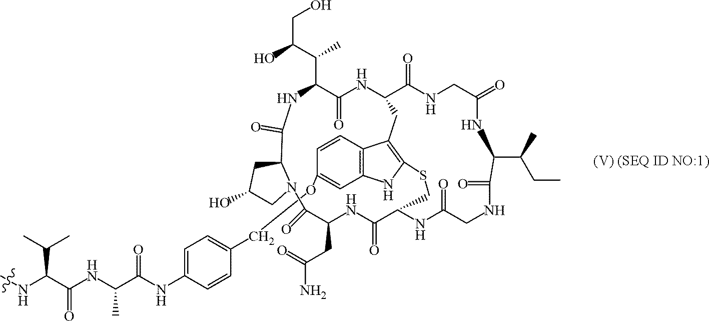 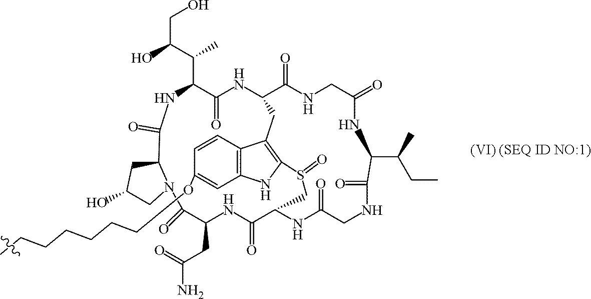 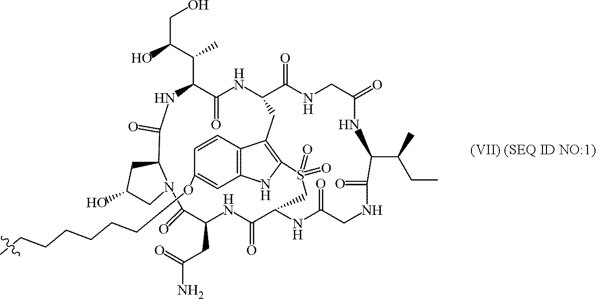 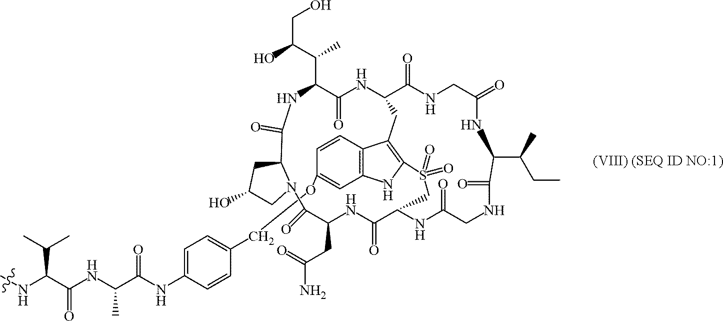 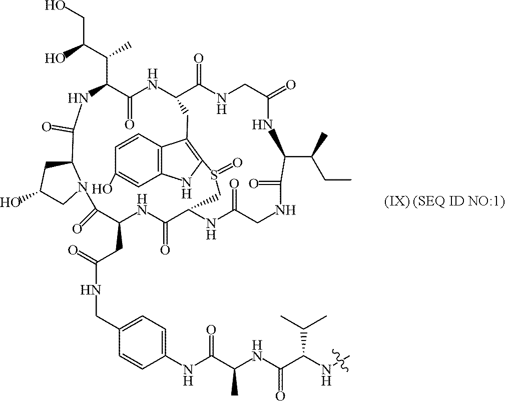 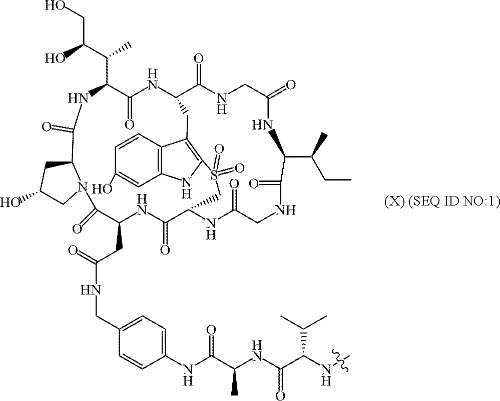 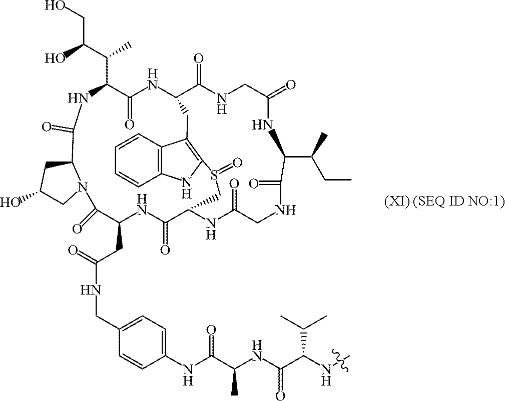
.

9. The method according to claim 8, wherein the first and/or second ADC comprises a compound according to any one of formulae (XII) to (XXII): 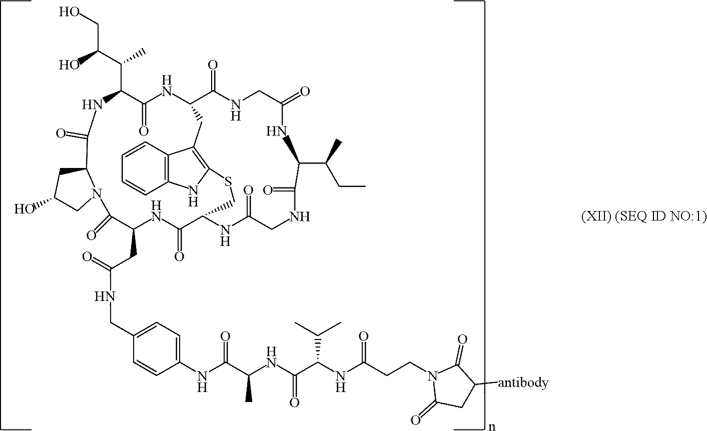 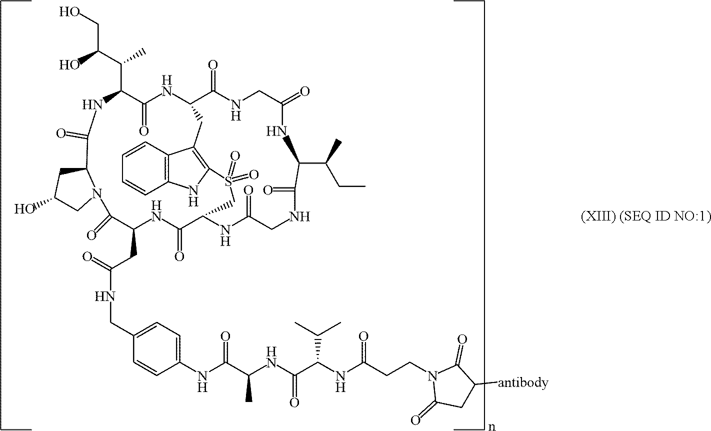 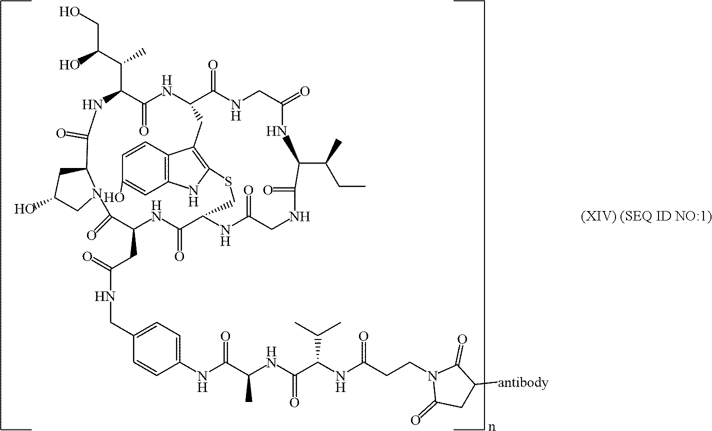 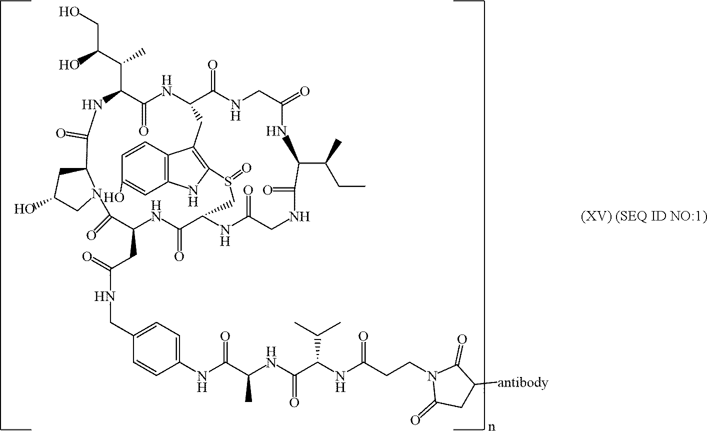 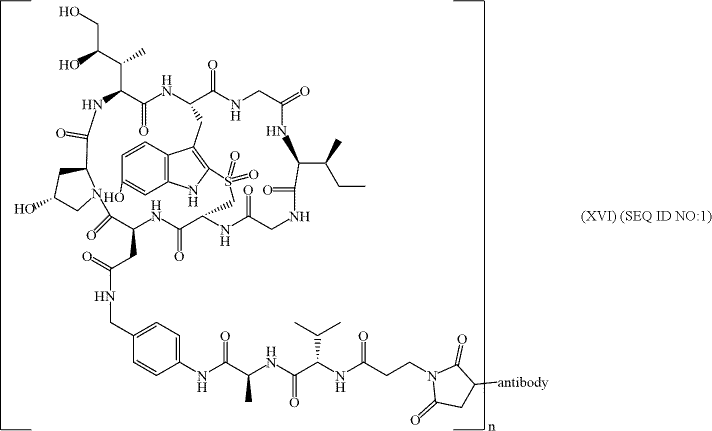 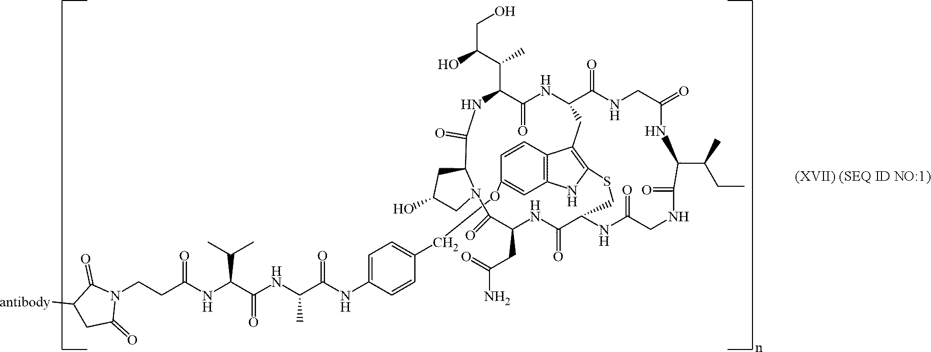 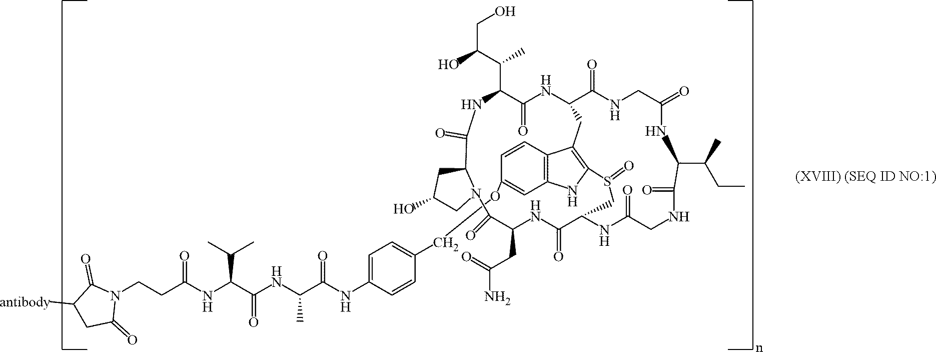 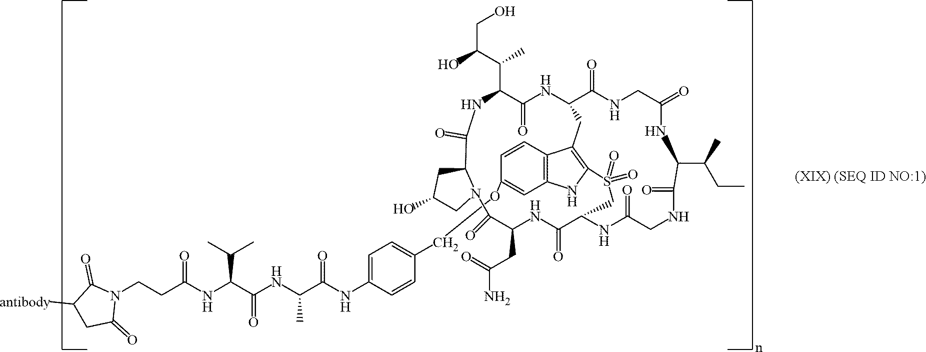 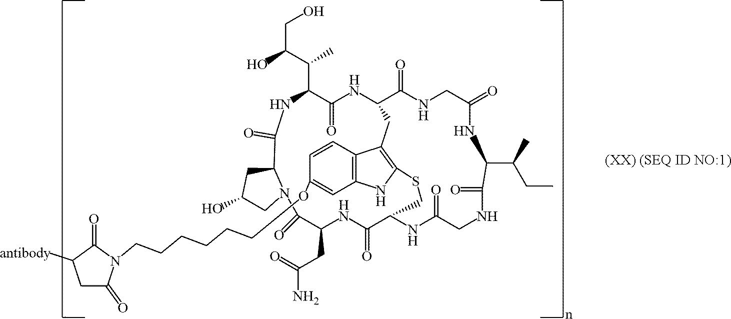 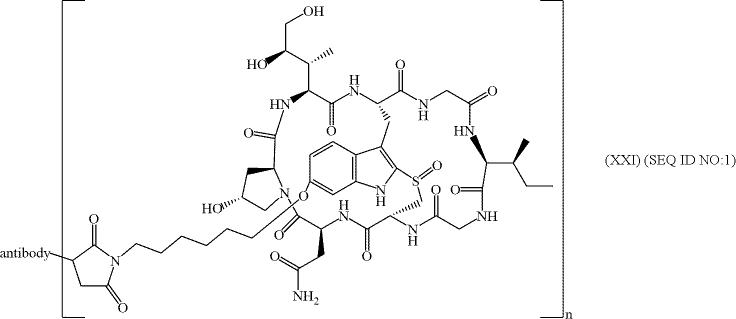 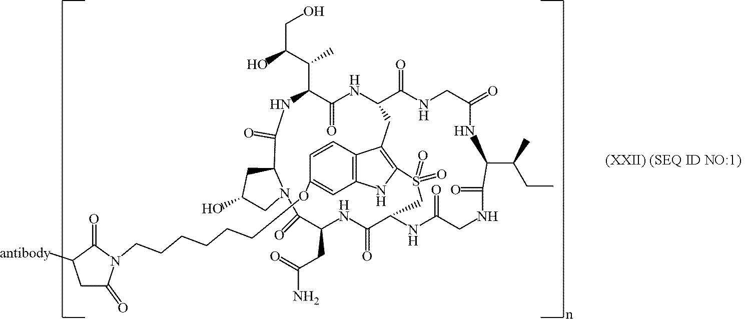
wherein n is from about 1 to about 10.

10. The method according to claim 1, wherein the antibody-drug-conjugate of said first does not specifically bind to a cell surface antigen, tumor associated antigen or tumor antigen of said patient.

11. The method according to claim 1, wherein said ADC of said second dose specifically binds to cell surface antigen, tumor associated antigen or tumor antigen of said patient.

12. The method according to claim 1, wherein the first and/or second antibody-drug-conjugate comprises a chimeric antibody.

13. The method according to claim 1, wherein the first and/or second antibody-drug-conjugate comprises a humanized antibody.

14. The method according to claim 1, wherein the first and/or second antibody-drug-conjugate comprises a human antibody.

15. The method according to claim 14, wherein the first and/or second antibody is selected from an IgG1, IgG2, or IgG4 isotype.

16. The method according to claim 15, wherein the first and/or second antibody is an IgG1 isotype.

17. The method according to claim 16, wherein the first and/or second antibody is a recombinant antibody.

18. The method according to claim 17, wherein the first and/or second antibody comprises at least one amino acid substitution in its Fc region.

19. The method according to claim 18, wherein the first and/or second antibody comprises at least one amino acid substitution selected from the group of L234A, L234G, L234T, L234Q, L234H; L235A, L235D, L235G, L235H, L235V; L236I, L236N, L236P,L236R, L236G, G237R, G237S,G237T,G237D,G237I, P238A, P238G,P238S,P238T, D265C, P329A,P329G,P329I, P329L,P329M, P329T (according to EU numbering).

20. The method according to claim 19, wherein the first and/or second antibody comprises at least two amino acid substitutions selected from L234A/L235A, L234A/L235D, L234A/L235G, L234A/L235H, L234A/L235V, L234G/L235A, L234G/L235D, L234G/L235G, L234G/L235H, L234G/L235V,L234T/L235A, L234T/L235D, L234T/L235G, L234T/L235H, L234T/L235V, L234A/G237R, L234A/G237S, L234A/G237T, L234A/G237D, L234A/G237I, L234G/G237R, L234A/G237S, L234A/G237T, L234A/G237D, L234A/G237I, L234T/G237R, L234T/G237S, L234T/G237T, L234T/G237D, L234T/G237I, L234A/P238A, L234A/P238G, L234G/P238A, L234G/P238G, L234T/P238A, L234T/P238G, L234A/D265C, L234A/D265C, L234A/D265C, L234G/D265C, L234G/D265C, L234G/D265C, L234T/D265C, L234T/D265C, L234T/D265C, L235A/D265C, L235G/D265C, L235V/D265C (according to EU numbering).

21. The method according to claim 20, wherein the first and/or second antibody comprises at least three amino acid substitutions selected from L234A/L235A/D265C, L234A/L235D/D265C, L234A/L235G/D265C, L234A/L235H/D265C, L234A/L235V/D265C, L234G/L235A/D265C, L234G/L235D/D265C L234G/L235G/D265C, L234G/L235H/D265C, L234G/L235V/D265C, L234T/L235A/D265C, L234T/L235D/D265C, L234T/L235G/D265C, L234T/L235H/D265C, L234T/L235V/D265C.

22. The method according to claim 21, wherein the first and/or second antibody comprises at least three amino acid substitutions selected from L234A/L235A/D265C, L234A/L235D/D265C, L234A/L235G/D265C, L234G/L235A/D265C, L234G/L235D/D265C L234G/L235G/D265C, L234T/L235A/D265C, L234T/L235D/D265C, L234T/L235G/D265C.

23. The method according to claim 22, wherein the first and/or second antibody comprises the amino acid substitutions L234A/L235A/D265C, L234G/L235A/D265C, or L234T/L235A/D265C (according to EU numbering).

24. The method according to claim 23, wherein the first and/or second antibody comprises the amino acid substitutions L234A/L235A/D265C (according to EU numbering).

25. The method according to claim 1, wherein said first antibody-drug conjugate is administered between about 30 to about 15 days prior to the administration of a second dose of a second antibody-drug conjugate.

26. The method according to claim 1, wherein the first antibody-drug-conjugate is dosed at about 60% to about 100% of the therapeutically effective dose of said second antibody-drug conjugate.

27. An antibody-drug conjugate according to any one of formulae (XII) to (XXII) 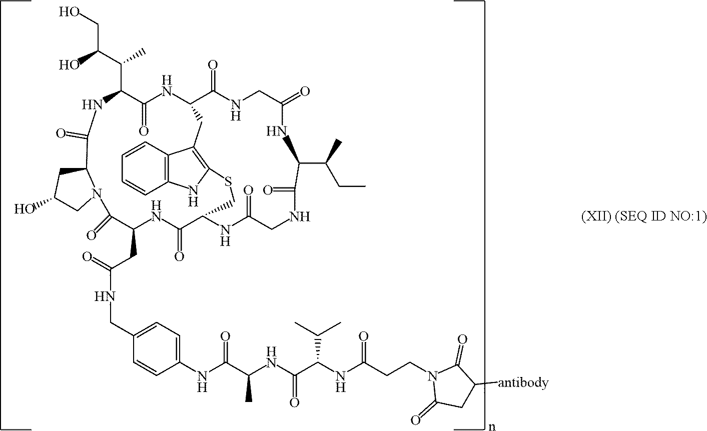 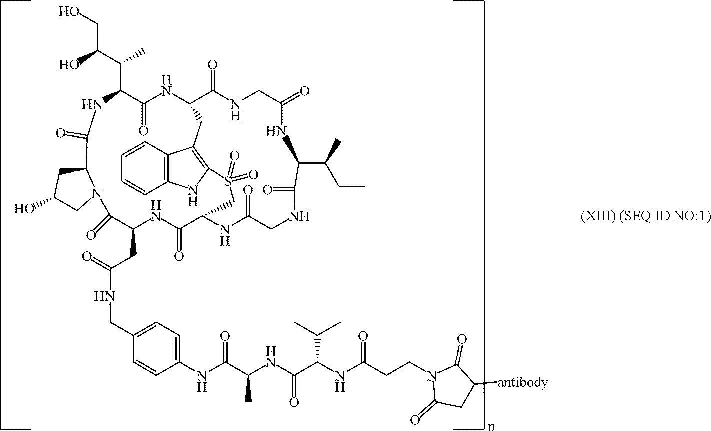 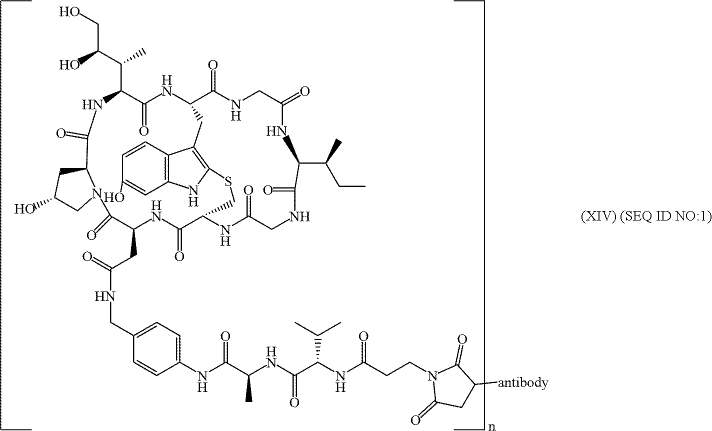 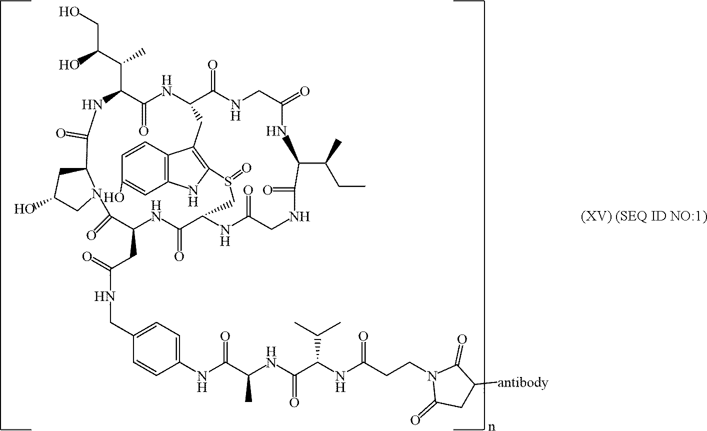 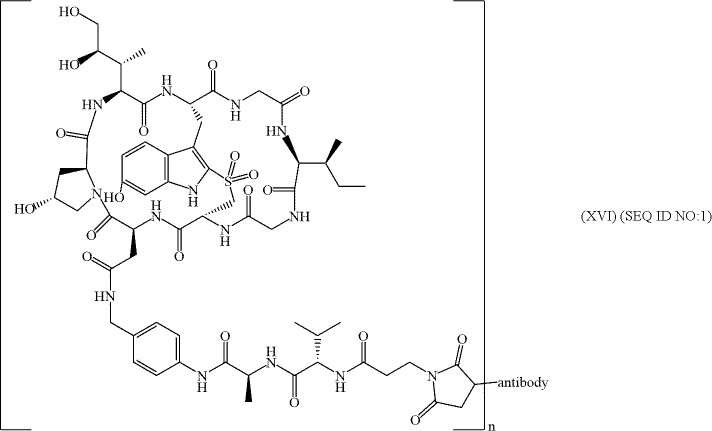 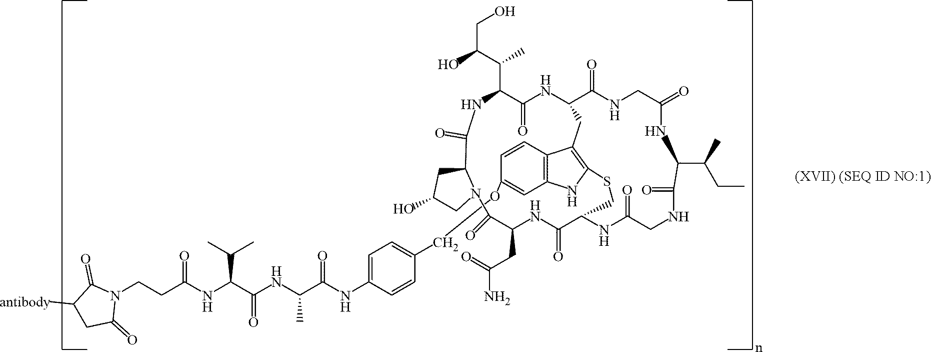 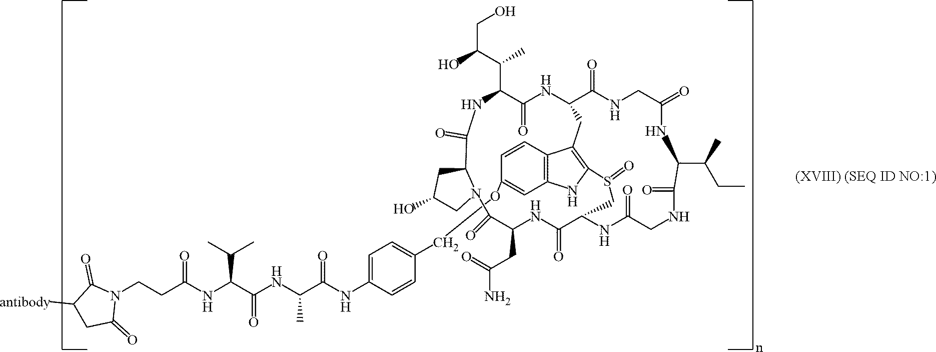 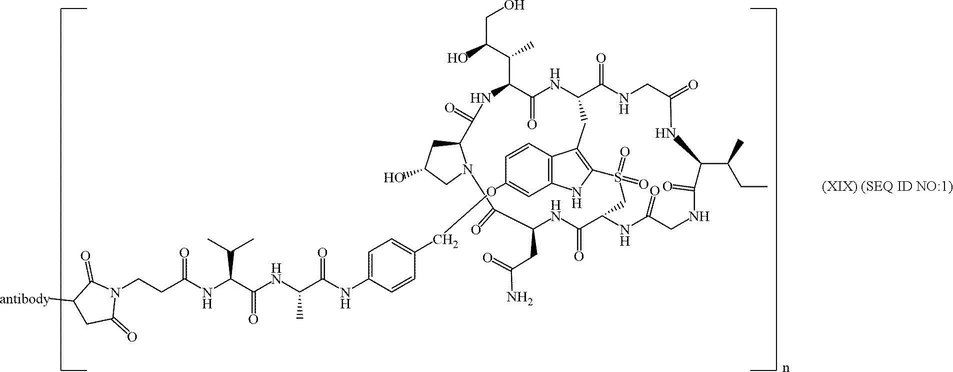 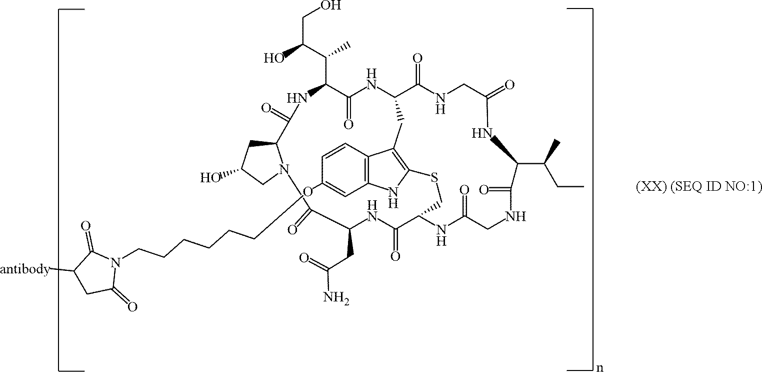 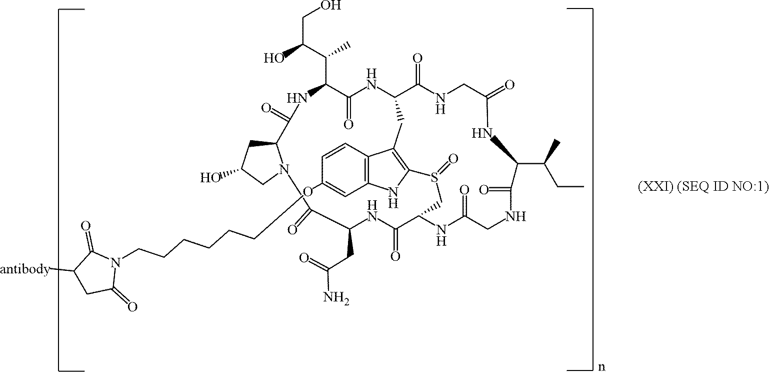 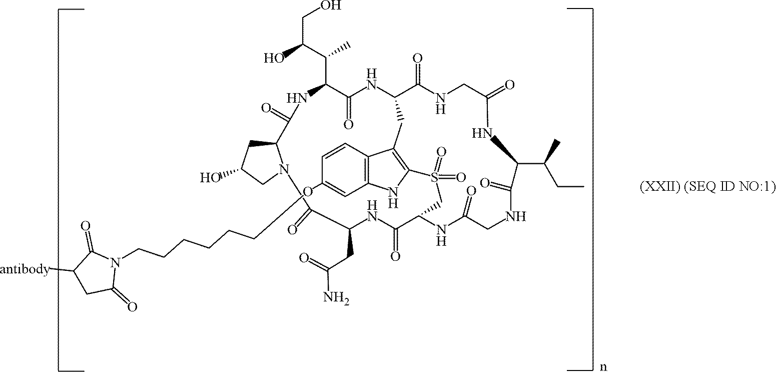
wherein n is from about 1 to about 10 for use in a method of increasing the therapeutic index according to claim 1.


SEQUENCE LISTING (2 sequences)

SEQ ID NO: 1 - protein, 8 aa, synthetic construct
  MOD_RES 2: note = 4Hyp
  REGION 1..8: note = circular peptide
NPIWGIGC

SEQ ID NO: 2 - protein, 8 aa, synthetic construct
  MOD_RES 2: note = 4Hyp
  REGION 1..8: note = circular peptide
DPIWGIGC